(19) United States
(12) Patent Application Publication
(10) Pub. No.: US 2026/0216325 A1
(43) Pub. Date: Jul. 30, 2026

(54) NOVEL IMIDAZOPYRIMIDINE COMPOUND AND USES THEREOF

(71) Applicants: The Children's Medical Center Corporation, Boston, MA (US); Dana-Farber Cancer Institute, Inc., Boston, MA (US)

(72) Inventors: David J. Dowling, Milton, MA (US); Ofer Levy, Cambridge, MA (US); Dheeraj Soni, Watertown, MA (US); David A. Scott, Newton, MA (US)

(73) Assignees: The Children's Medical Center Corporation, Boston, MA (US); Dana-Farber Cancer Institute, Inc., Boston, MA (US)

(21) Appl. No.: 19/146,280

(22) PCT Filed: Jan. 8, 2024
(86) PCT No.: PCT/US2024/010632

§ 371 Date: Jul. 8, 2025

Related U.S. Application Data
(60) Provisional application No. 63/437,902, filed on Jan. 9, 2023.

(51) Int. Cl. A61K 39/39; A61K 9/00; A61K 31/519; A61K 39/00; A61K 39/145; A61K 39/215; A61K 39/385; A61P 31/16; C07D 487/04; C12N 7/00

(52) U.S. Cl. CPC A61K 39/39; A61K 9/0019; A61K 31/519; A61K 39/145; A61K 39/215; A61K 39/385; A61P 31/16; C07D 487/04; C12N 7/00; A61K 2039/545; A61K 2039/55; A61K 2039/55505; A61K 2039/575; A61K 2039/60; C12N 2760/16134; C12N 2760/16234; C12N 2770/20034

(57) ABSTRACT

The present disclosure provides Compound (1), and pharmaceutically acceptable salts, solvates, hydrates, polymorphs, co-crystals, tautomers, stereoisomers, isotopically labeled derivatives, and compositions thereof. Compound (1) is used as an enhancer and/or modifier of an immune response (e.g., innate and/or adaptive immune response), and is useful in treating and/or preventing a disease, as an adjuvant in a vaccine for a disease, (e.g., a proliferative disease, an inflammatory disease, an autoimmune disease, an infectious disease, an allergy, a fibrotic disease, a cardiovascular disease, a graft rejection, graft-versus-host disease, chronic disease, or addiction), or as stand alone anti-infective or immune response modifying agents. Also provided in the present disclosure are vaccines, pharmaceutical compositions, kits, methods, and uses including or using Compound (1).

### RELATED APPLICATIONS

This application claims priority under 35 U.S.C. § 119(e) to U.S. Provisional Application, U.S. Ser. No. 63/437,902, filed Jan. 9, 2023, which is incorporated herein by reference in its entirety.

### FEDERALLY SPONSORED RESEARCH

This invention was made with government support under Contract Numbers 75N93019C00044 and HHSN272201400052C, awarded by the National Institutes of Health. The Government has certain rights in the invention.

### BACKGROUND

Vaccination remains one of the most cost-effective health interventions available, significantly reducing morbidity, and preventing 4-5 million deaths every year from vaccine-preventable infectious diseases (VPID) such as diphtheria, tetanus, pertussis, measles, influenza, and SARS-CoV-2. In addition, vaccines can be employed for non-infectious indications such as prevention of cancer (e.g., human papilloma virus vaccine and hepatitis B vaccine) with on-going discovery and development of vaccines for drug (e.g., opioid) overdose. Contemporary vaccine strategies generally moved away from live attenuated vaccines with a greater focus on target defined antigens, employing a broad set of platforms, including purified recombinant proteins and genetic delivery. Although this approach has lowered reactogenicity of many clinically licensed formulations, vaccine efficacy and durability of protection may lag, especially in vulnerable populations such as the very young and older adults with distinct and generally weaker immunity, due to insufficient innate immune stimulation important to enhancing the magnitude, breadth and durability of vaccine immunogenicity. Adjuvants can enhance vaccine responses by activating pattern-recognition receptors (PRRs) and/or by modulating antigen pharmacokinetics. Indeed, many first-generation 20^{th} century vaccines, consisting of live attenuated or inactivated vaccines, expressed immune-stimulating components that activate PRRs and are therefore “self-adjuvanted”. Multi-disciplinary investigations of innate immunity and systems vaccinology have further informed discovery and development of novel adjuvants, which may also have utility in developing adjuvanted vaccines against SARS-CoV-2 and future pandemics. Thus, there is a need to identify compounds capable of modifying human immune responses that may act as therapeutics on their own and/or as adjuvants.

### SUMMARY

Although Toll-like receptor 7 and 8 (TLR7/8) agonists have a history of efficacy in pre-clinical and clinical settings, the present disclosure stems from the recognition that known small molecule TLR7/8 agonists such as R848 (resiquimod) have high reactogenicity, limiting their use in development of new adjuvanted vaccines. Thus, novel TLR7/8 agonists which promote targeted delivery and avert systemic inflammation, but still activate the innate and adaptive immune systems, are desirable. Novel small molecule adjuvants offer significant advantages such as targeted activation of antigen presenting cells (APCs), increased stability of small molecules for longer duration, potential dose-sparing effects, improved reactogenicity profiles, long-term safety, and efficacy.

Accordingly, some aspects of the present disclosure are based on the finding that a novel imidazopyrimidine compound (i.e., Compound (1)) possesses surprisingly exceptional properties that render it useful as both a single-agent therapeutic and as an adjuvant in vaccines. For example, the compound may enhance Th1-skewed immune responses toward influenza antigens in vivo in a prime-boost immunization schedule. Furthermore, the compound may be useful in vaccines for indications requiring enhancement of Th1-polarized immune responses.

Accordingly, provided herein is a compound of formula:

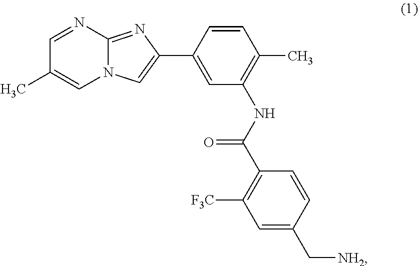 [SMILES: Cc1cnc2nc(-c3ccc(C)c(NC(=O)c4ccc(CN)cc4C(F)(F)F)c3)cn2c1]

and pharmaceutically acceptable salts, solvates, hydrates, polymorphs, co-crystals, tautomers, stereoisomers, and isotopically labeled derivatives thereof. Compound (1) is useful in enhancing and/or modifying human immune responses, including innate and adaptive immune responses. In some embodiments, Compound (1) is used as an adjuvant in vaccines for disease (e.g., a proliferative disease, an inflammatory disease, an autoimmune disease, an infectious disease, an allergy, a fibrotic disease, a cardiovascular disease, a graft rejection, graft-versus-host disease, chronic disease, or addiction), or as a stand alone anti-infective or immune response modifying agent (e.g., single agent), in a subject in need thereof. In some aspects, using Compound (1) as a vaccine adjuvant enables effective immunization in vulnerable populations (e.g., neonates, infants, older adults, or immunocompromised individuals).

In another aspect, provided are pharmaceutical compositions including Compound (1) and optionally a pharmaceutically acceptable excipient. In certain embodiments, a pharmaceutical composition described herein includes a therapeutically or prophylactically effective amount of Compound (1). In certain embodiments, a pharmaceutical composition described herein further comprises an additional pharmaceutical agent. The pharmaceutical compositions may be useful as enhancers and/or modifiers of an immune response (e.g., innate and/or adaptive immune response), and/or adjuvants in a vaccine for a disease, (e.g., a proliferative disease, an inflammatory disease, an autoimmune disease, an infectious disease, an allergy, a fibrotic disease, a cardiovascular disease, a graft rejection, graft-versus-host disease, chronic disease, or addiction), or as stand alone anti-infective or immune response modifying agents.

In another aspect, provided are compositions comprising an antigen and Compound (1). In some embodiments, the composition is a vaccine composition. In some embodiments, Compound (1) is an adjuvant. In another aspect, provided are vaccines comprising an antigen and Compound (1).

In another aspect, provided are kits including a container with a compound, composition, or vaccine described herein. A kit described herein may include a single dose or multiple doses of the compound, compositions, or vaccines. The described kits may be useful in enhancing an immune response (e.g., innate and/or adaptive immune response) in a subject, biological sample, tissue, or cell, in treating a disease (e.g., a proliferative disease, an inflammatory disease, an autoimmune disease, an infectious disease, an allergy, a fibrotic disease, a cardiovascular disease, a graft rejection, graft-versus-host disease, chronic disease, or addiction) in a subject in need thereof, and/or in preventing a disease (e.g., a proliferative disease, an inflammatory disease, an autoimmune disease, an infectious disease, an allergy, a fibrotic disease, a cardiovascular disease, a graft rejection, graft-versus-host disease, chronic disease, or addiction) in a subject in need thereof. In certain embodiments, a kit described herein further includes instructions for using the compound, compositions, or vaccines included in the kit.

In another aspect, the present disclosure provides methods of enhancing an immune response (e.g., innate and/or adaptive immune response) in a subject in need thereof, the methods comprising administering to the subject a therapeutically effective amount of a compound, composition, or vaccine described herein.

In another aspect, the present disclosure provides methods of enhancing an immune response (e.g., innate and/or adaptive immune response) to an antigen in a subject in need thereof, the methods comprising administering to the subject a therapeutically effective amount of a compound, composition, or vaccine described herein.

In another aspect, the present disclosure provides methods of treating a disease (e.g., a proliferative disease, an inflammatory disease, an autoimmune disease, an infectious disease, an allergy, a fibrotic disease, a cardiovascular disease, a graft rejection, graft-versus-host disease, chronic disease, or addiction) in a subject in need thereof, the methods comprising administering to the subject a therapeutically effective amount of a compound, composition, or vaccine described herein. In another aspect, the present disclosure provides methods of preventing a disease in a subject in need thereof, the methods comprising administering to the subject a prophylactically effective amount of a compound, composition, or vaccine described herein.

In another aspect, provided are methods of vaccinating a subject in need thereof, the method comprising administering to the subject an effective amount of a compound, composition, or vaccine described herein.

In yet another aspect, the present disclosure provides a compound, compositions, and/or vaccines described herein for use in a method of the disclosure (e.g., enhancing an immune response (e.g., innate and/or adaptive immune response), a method of treating and/or preventing a disease (e.g., a proliferative disease).

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

The term “pharmaceutically acceptable salt” refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”. In some embodiments, a stereoisomer may be an atropisomer (stereoisomers arising because of hindered rotation about a single bond, where energy differences due to steric strain or other contributors create a barrier to rotation that is high enough to allow for isolation of individual conformers).

The term “polymorphs” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “small molecule” refers to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or older adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. A “patient” refers to a human subject in need of treatment of a disease or disorder. The subject may also be a plant. In certain embodiments, the plant is a land plant. In certain embodiments, the plant is a non-vascular land plant. In certain embodiments, the plant is a vascular land plant. In certain embodiments, the plant is a seed plant. In certain embodiments, the plant is a cultivated plant. In certain embodiments, the plant is a dicot. In certain embodiments, the plant is a monocot. In certain embodiments, the plant is a flowering plant. In some embodiments, the plant is a cereal plant, e.g., maize, corn, wheat, rice, oat, barley, rye, or millet. In some embodiments, the plant is a legume, e.g., a bean plant, e.g., soybean plant. In some embodiments, the plant is a tree or shrub.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The terms “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., considering a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. When an effective amount of a composition is referred herein, it means the amount is prophylactically and/or therapeutically effective, depending on the subject and/or the disease to be treated. Determining the effective amount or dosage is within the abilities of one skilled in the art.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a compound described herein is an amount effective to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

An “adjuvant” refers to a pharmacological or immunological agent that modifies the effect of other agents, for example, of an antigen in a vaccine. Adjuvants are typically included in vaccines to enhance the recipient subject's immune response to an antigen. The use of adjuvants allows the induction of a greater immune response in a subject with the same dose of antigen, or the induction of a similar level of immune response with a lower dose of injected antigen. Adjuvants that are known to those of skill in the art, include, without limitation: aluminum salts, liposomes, lipopolysaccharide (LPS), molecular cages for antigen, components of bacterial cell walls, endocytosed nucleic acids such as double-stranded RNA (dsRNA), single-stranded DNA (ssDNA), and unmethylated CpG dinucleotide-containing DNA. Adjuvants are thought to function in several ways, for example, but not limited to, increasing the surface area of antigen, prolonging the retention of the antigen in the body thus allowing time for the lymphoid system to have access to the antigen, slowing the release of antigen, targeting antigen to macrophages, engaging pattern recognition receptors (PRRs) such as Toll-like receptors (TLRs) to activate leukocytes such as antigen-presenting cells (e.g., monocytes, macrophages, and/or dendritic cells), or otherwise eliciting broad activation of the cells of the immune system see, e.g., H. S. Warren et al, Annu. Rev. Immunol., 4:369 (1986), incorporated herein by reference.

The ability of an adjuvant to induce and increase a specific type of immune response and the identification of that ability is thus a key factor in the selection of particular adjuvants for vaccine use against a particular pathogen. Adjuvants that are known to those of skill in the art, include, without limitation: aluminum salts (referred to herein as “alum”), liposomes, lipopolysaccharide (LPS) or its derivatives such as monophosphoryl lipid A (MPLA), molecular cages for antigen, components of bacterial cell walls, endocytosed nucleic acids such as double-stranded RNA (dsRNA), single stranded RNA (ssRNA), single-stranded DNA (ssDNA), and unmethylated CpG dinucleotide-containing DNA. Typical adjuvants include water and oil emulsions, e.g., Freund's adjuvant and MF59, and chemical compounds such as aluminum hydroxide or alum. At present, currently licensed vaccines in the United States contain only a limited number of adjuvants, such as alum that enhances production of T helper 2 (Th2) cells and MPLA which activates innte immunity via Toll-like receptor 4 (TLR4). Many of the most effective adjuvants include bacteria or their products, e.g., microorganisms such as the attenuated strain of Mycobacterium bovis, Bacillus Calmette-Guérin (BCG); microorganism components, e.g., alum-precipitated diphtheria toxoid, bacterial lipopolysaccharide and endotoxins or their derivatives such as MPLA.

The term “infectious disease” refers to an illness caused by a pathogenic biological agent that results from transmission from an infected person, animal, or reservoir to a susceptible host, either directly or indirectly, through an intermediate plant or animal host, vector, or inanimate environment. See Last J M. ed. A dictionary of epidemiology. 4th ed., New York: Oxford University Press, 1988. Infectious disease is also known as transmissible disease or communicable disease. In certain embodiments, infectious diseases may be asymptomatic for much or even all of their course in a given host. Infectious pathogens include some viruses, bacteria, fungi, protozoa, multicellular parasites, and aberrant proteins known as prions.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis”, “metastatic”, or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, hematological malignancies. Additional exemplary cancers include, but are not limited to, lung cancer (e.g., bronchogenic carcinoma, small cell lung cancer (SCLC), non-small cell lung cancer (NSCLC), adenocarcinoma of the lung); kidney cancer (e.g., nephroblastoma, a.k.a. Wilms' tumor, renal cell carcinoma); acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; liver cancer (e.g., hepatocellular cancer (HCC), malignant hepatoma); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). In some embodiments, the cancer treated using the composition and methods of the present disclosure is melanoma.

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

A “chronic disease” refers to a disease lasting for three or more months. Exemplary chronic diseases include, but are not limited to, arthritis, cardiovascular disease such as heart disease, stroke, cancer (e.g., breast cancer or colon cancer), chronic respiratory diseases, diabetes, epilepsy, seizures, obesity, and oral health problems.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1E show that Compound (1) has enhanced potency in vitro and adjuvanticity in vivo. FIG. 1A: Chemical structures of PVP-037.1 and PVP-037.2. Compound (1) is PVP-037.2. FIG. 1B: PVP-037.2 was identified as the most potent molecule. TNF production after stimulation of human adult PBMCs with R848 (resiquimod), PVP-037.1, and PVP-037.2 for 18 hrs at various concentrations. PVP-037.2 augmented TNF production via human PBMC in vitro assays as compared to PVP-037.1 and R848. FIG. 1C: To evaluate PK (drug serum levels) C57BL/6J mice, at 6-8 weeks of age and weighing 20-30 grams, received a single administration of each formulation was intravenously, employing a total volume of 20 μl per mouse. Blood/serum samples were collected (tail vein) from the same mouse pre- (0 min) and 0.1, 0.25, 0.5, 2-, 4-, 6- and 8-hours post-administration. An inactive analog (Cpd 02-139-1) was also evaluated. Left panel indicates the plasma concentration over time. Right panel indicates % plasma concentration relative to first draw baseline. FIG. 1D: 6-8 weeks old C57BL/6 adult mice were injected IM prime (Day 0) with saline, rHA alone, rHA admixed with PVP-037.1 or PVP-037.2. Ab titers for rHA-specific IgG isotypes were measured by ELISA 28 days post-immunization. FIG. 1E: 6-8 weeks old C57BL/6 adult mice were injected IM prime (Day 0) with saline, WT spike protein alone, spike admixed with PVP-037.2 (100 nmoles/mice). Ab titers for WT spike-specific IgG, IgG1 or IgG2c were measured by ELISA 28 days post-immunization. hACE2-RBD (WT) inhibition rate was determined 42 days post-immunization. For in vitro results are presented as line graph (N=5). * p<0.05, ** p<0.01, *** p<0.001, **** p<0.0001 of small molecules vs DMSO by repeated measure one-way ANOVA of Log-transformed data. For in vivo immunogenicity results are presented as box and whisker plots of 10-20 mice/group (except, sVNT N=5). * p<0.05, ** p<0.01 of rHA+PVP molecules vs rHA by repeated measure one-way ANOVA of Log-transformed data.

FIGS. 2A-2C show that Compound (1) demonstrates TLR7-dependent enhancement of Th1-polarizing adjuvanticity. FIG. 2A: PBMCs from adult human donors were incubated with inhibitory (i)ODNs (TLR7/8/9 antagonists consisting of short single-stranded oligodeoxynucleotides), either ODN 2088 (TLR7/8/9 antagonist) or ODN 20958 (TLR7 antagonist) for 1 hr. After inhibition with iODNs, cells were stimulated with CL264 (TLR7A) or PVP.037.2, and supernatants were harvested after 20-24 hrs. TNF production was assayed in the supernatants via ELISA (N=6 donors). FIG. 2B: Bone-marrow were isolated from WT or TLR7^{-/-} mice. The cells were cultured in growth media containing mM-CSF (20 ng/ml). After 7 days, adherent cells (Bone-marrow derived macrophages; BMDM) were harvested and were stimulated with 33 μM of CL264 (TLR7A) (2^{nd} from right) or R848 (TLR7/8A) (3^{rd} from right), 100 ng/mL LPS (TLR4A) (farthest right), 100 μM PVP-037.1 (2^{nd} from left) or PVP-037.2 (3^{rd} from left). The supernatants were collected after 20-24 hrs and TNF production measured via ELISA (N=5 samples). FIG. 2C: 6-8 weeks old WT or TLR7^{-/-} mice were injected IM prime (Day 0) with saline, rHA alone, and rHA admixed with 100 nmoles of PVP-037.2. Ab titers for rHA-specific IgG or IgG2c were measured by ELISA 28 days post-immunization. (N=5 samples)

FIG. 3 shows that young mice vaccinated with Compound (1) maintain normal weight gains. Young C57BL/6 adult mice (6-8 weeks old) were vaccinated IM (Day 0) with saline, rHA alone (1 μg antigen from Flublok 2017/18), rHA admixed with the PVP-037.2 (100 nmol). Body weight was measured up to 3 days post vaccination and presented as percent weight change as compared to day 0 (N=5). Results represent mean±SEM. No significant differences were observed between treatment groups.

FIG. 4 shows that Compound (1) demonstrates robust adjuvanticity across mouse strains. 6-8 weeks old Balb/c adult mice were vaccinated IM (Day 0) with saline (bottom curve), Spike alone (1 μg antigen from wild type SARS-CoV-2) (middle curve), Spike admixed with the PVP-037.2 (100 nmol) (top curve). Ab titers for Spike-specific IgG and IgG1 were measured by ELISA on Days 0, 14 and 28 post-immunization (N=10). Results represent mean±SEM. For comparison at individual days, repeated measures two-way ANOVA, *p<0.05, **p<0.01, ***p<0.01.

### DETAILED DESCRIPTION OF CERTAIN EMBODIMENTS

Described herein is a compound of formula:

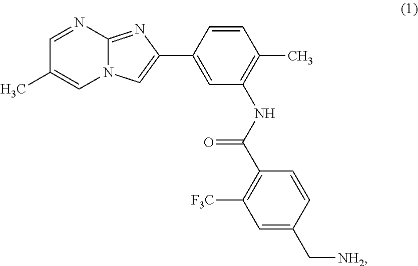 [SMILES: Cc1cnc2nc(-c3ccc(C)c(NC(=O)c4ccc(CN)cc4C(F)(F)F)c3)cn2c1]

and pharmaceutically acceptable salts, solvates, hydrates, polymorphs, co-crystals, tautomers, stereoisomers, and isotopically labeled derivatives thereof. In certain embodiments, Compound (1) refers to a pharmaceutically acceptable salt of Compound (1). In certain embodiments, Compound (1) refers to a solvate of Compound (1). In certain embodiments, Compound (1) refers to a hydrate of Compound (1). In certain embodiments, Compound (1) refers to a polymorph of Compound (1). In certain embodiments, Compound (1) refers to a co-crystal of Compound (1). In certain embodiments, Compound (1) refers to a tautomer of Compound (1). In certain embodiments, Compound (1) refers to a stereoisomer of Compound (1). In certain embodiments, Compound (1) refers to an isotopically labeled derivative of Compound (1).

Compound (1) possesses surprisingly exceptional properties (e.g., robust immunomodulatory activity) rendering it useful as both a single-agent therapeutic and as an adjuvant in vaccines. For example, Compound (1) is stable in compositions, induces robust cytokine production in vitro in antigen presenting cells both in bone-marrow derived dendritic cells (BMDCs) and bone-marrow derived macrophages (BMDMs), and enhances immunogenicity of vaccinal antigens in vivo. Compound (1) also possesses improved solubility, enhanced potency, and improved pharmacokinetic properties relative to known imidazopyrimidine compounds such as PVP-037.1.

As such, Compound (1) is an enhancer and/or modifier of an immune response (e.g., innate and/or adaptive immune response), and/or an adjuvant in a vaccine for a disease (e.g., a proliferative disease, an inflammatory disease, an autoimmune disease, an infectious disease, an allergy, a fibrotic disease, a cardiovascular disease, a graft rejection, graft-versus-host disease, chronic disease, or addiction), or as a stand alone (e.g., single agent) anti-infective or immune response modifying agent. Compound (1) is useful in treating or preventing a disease (e.g., a proliferative disease, an inflammatory disease, an autoimmune disease, an infectious disease, an allergy, a fibrotic disease, a cardiovascular disease, a graft rejection, graft-versus-host disease, chronic disease, or addiction) in a subject in need thereof. Also provided are pharmaceutical compositions, vaccines, kits, and uses including Compound (1).

### Pharmaceutical Compositions, Kits, and Administration

The present disclosure provides pharmaceutical compositions comprising Compound (1), or a pharmaceutically acceptable salt, solvate, hydrate, polymorph, co-crystal, tautomer, stereoisomer, or isotopically labeled derivative, and optionally a pharmaceutically acceptable excipient.

In certain embodiments, Compound (1) is provided in an effective amount in the pharmaceutical composition. In certain embodiments, the effective amount is a therapeutically effective amount. In certain embodiments, the effective amount is a prophylactically effective amount. In certain embodiments, a therapeutically effective amount is an amount effective for enhancing an immune response (e.g., innate and/or adaptive immune response). In certain embodiments, a therapeutically effective amount is an amount effective for treating a disease (e.g., proliferative disease). In certain embodiments, a therapeutically effective amount is an amount effective for serving as an adjuvant in a vaccine for a disease, (e.g., a proliferative disease, an inflammatory disease, an autoimmune disease, an infectious disease, an allergy, a fibrotic disease, a cardiovascular disease, a graft rejection, graft-versus-host disease, chronic disease, or addiction), or as stand alone anti-infective or immune response modifying agents. In certain embodiments, a prophylactically effective amount is an amount effective for preventing a disease (e.g., a proliferative disease, an inflammatory disease, an autoimmune disease, an infectious disease, an allergy, a fibrotic disease, a cardiovascular disease, a graft rejection, graft-versus-host disease, chronic disease, or addiction). In certain embodiments, a prophylactically effective amount is an amount effective for enhancing an immune response (e.g., innate and/or adaptive immune response), and preventing a disease (e.g., a proliferative disease, an inflammatory disease, an autoimmune disease, an infectious disease, an allergy, a fibrotic disease, a cardiovascular disease, a graft rejection, graft-versus-host disease, chronic disease, or addiction).

In certain embodiments, the effective amount is an amount effective for enhancing an immune response (e.g., innate and/or adaptive immune response) by at least 10%, at least 20%, at least 30%, at least 40%, at least 50%, at least 60%, at least 70%, at least 80%, at least 90%, at least 95%, or at least 98%. In certain embodiments, the effective amount is an amount effective for enhancing an immune response (e.g., innate and/or adaptive immune response) by not more than 10%, not more than 20%, not more than 30%, not more than 40%, not more than 50%, not more than 60%, not more than 70%, not more than 80%, not more than 90%, not more than 95%, or not more than 98%.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. The use of the compounds described herein in veterinary vaccine is also within the scope of the present disclosure. “A companion animal,” as used herein, refers to pets and other domestic animals. Non-limiting examples of companion animals include dogs and cats; livestock such as horses, cattle, pigs, sheep, goats, and chickens; and other animals such as mice, rats, guinea pigs, and hamsters. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat, guinea pig, and hamster), dog, pig, rabbit, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the cell is present in vitro. In certain embodiments, the cell is present in vivo.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

The compound and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical, mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or composition is administered intradermally, intramuscularly, intravaginally, intravenously, intranasally, orally, subcutaneously, transdermally, topically, and/or sublingually. In certain embodiments, the compound or composition is administered as a prophylactic. In certain embodiments, the compound or composition is administered as a combination therapy with another immunomodulatory agent, an immunomodulating antibody, an immunomodulating biologic, or an inhibitor of molecular pathways that limits immune responses. In certain embodiments, the immunomodulatory agent is a pattern recognition receptor agonist (e.g., an Alum, or a Toll-like receptor (TLR) Agonist). In certain embodiments, the immunomodulating antibody or immunomodulating biologic is a cytokine, chemokine or colony stimulating factor.

A compound or composition, as described herein, can be administered in combination with one or more additional pharmaceutical agents (e.g., therapeutically and/or prophylactically active agents). The compound or compositions can be administered in combination with additional pharmaceutical agents that improve their activity (e.g., activity (e.g., potency and/or efficacy) in treating a disease in a subject in need thereof, in preventing a disease in a subject in need thereof, in enhancing an immune response (e.g., innate and/or adaptive immune response) in a subject, biological sample, tissue, or cell), serving as an adjuvant in a vaccine for a disease (e.g., a proliferative disease, an inflammatory disease, an autoimmune disease, an infectious disease, an allergy, a fibrotic disease, a cardiovascular disease, a graft rejection, graft-versus-host disease, chronic disease, or addiction), improve bioavailability, improve safety, reduce drug resistance, reduce and/or modify metabolism, inhibit excretion, and/or modify distribution in a subject, biological sample, tissue, or cell, or as stand alone anti-infective or immune response modifying agents. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compounds and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., a proliferative disease, an inflammatory disease, an autoimmune disease, an infectious disease, an allergy, a fibrotic disease, a cardiovascular disease, a graft rejection, graft-versus-host disease, chronic disease, or addiction). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, and a combination thereof. In certain embodiments, the additional pharmaceutical agent is an anti-proliferative agent (e.g., anti-cancer agent). In certain embodiments, the additional pharmaceutical agent is an anti-leukemia agent. In certain embodiments, the additional pharmaceutical agent is an anti-lymphoma agent. In certain embodiments, the additional pharmaceutical agent is ABITREXATE (methotrexate), ABRAXANE (paclitaxel albumin-stabilized nanoparticle formulation), ABVD, ABVE, ABVE-PC, ADCETRIS (brentuximab vedotin), ADRIAMYCIN RDF (doxorubicin hydrochloride), AMBOCHLORIN (chlorambucil), ARRANON (nelarabine), AC, AC-T, ADE, ADRIAMYCIN PFS (doxorubicin hydrochloride), ADRUCIL (fluorouracil), AFINITOR (everolimus), AFINITOR DISPERZ (everolimus), ALDARA (imiquimod), ALIMTA (pemetrexed disodium), AREDIA (pamidronate disodium), ARIMIDEX (anastrozole), AROMASIN (exemestane), ARZERRA (ofatumumab), AVASTIN (bevacizumab), BEACOPP, BECENUM (carmustine), BELEODAQ (belinostat), BEP, BICNU (carmustine), BLENOXANE (bleomycin), BOSULIF (bosutinib), BUSULFEX (busulfan), CAF, CAMPATH (alemtuzumab), CLOFAREX (clofarabine), CLOLAR (clofarabine), CVP, CAMPTOSAR (irinotecan hydrochloride), CAPOX, CAPRELSA (vandetanib), CARBOPLATIN-TAXOL, CARMUBRIS (carmustine), CASODEX (bicalutamide), CEENU (lomustine), CERUBIDINE (daunorubicin hydrochloride), CERVARIX (recombinant HPV bivalent vaccine), CHOP, CLAFEN (cyclophosphamide), CMF, COMETRIQ (cabozantinib-s-malate), COPP, COPP-ABV, CVP, COSMEGEN (dactinomycin), CYFOS (ifosfamide), CYRAMZA (ramucirumab), CYTOSAR-U (cytarabine), CYTOXAN (cyclophosphamide), DACOGEN (decitabine), DEGARELIX, DEPOCYT (liposomal cytarabine), DTIC-DOME (dacarbazine), DOXIL (doxorubicin hydrochloride liposome), DOXORUBICIN HYDROCHLORIDE, DOX-SL (doxorubicin hydrochloride liposome), DTIC-DOME (dacarbazine), EFUDEX (fluorouracil), ELLENCE (epirubicin hydrochloride), ELOXATIN (oxaliplatin), EPOCH, ERBITUX (cetuximab), ERIVEDGE (vismodegib), ERWINAZE (Asparaginase Erwinia Chrysanthemi), ETOPOPHOS (etoposide phosphate), EVACET (doxorubicin hydrochloride liposome), EXXAR (tositumomab and iodine I 131 tositumomab), FARESTON (toremifene), FASLODEX (fulvestrant), FEC, FEMARA (letrozole), FLUDARA (fludarabine phosphate), FLUOROPLEX (fluorouracil), FOLEX (methotrexate), FOLEX PFS (methotrexate), FOLOTYN (pralatrexate), FOLFIRI, FOLFIRI-BEVACIZUMAB, FOLFIRI-CETUXIMAB, FOLFIRINOX, FOLFOX, FU-LV, GARDASIL (recombinant human papillomavirus (HPV) quadrivalent vaccine), GAZYVA (obinutuzumab), GEMCITABINE-CISPLATIN, GEMCITABINE-OXALIPLATIN, GEMZAR (gemcitabine hydrochloride), GILOTRIF (afatinib dimaleate), GLEEVEC (imatinib mesylate), GLIADEL (carmustine implant), GLIADEL WAFER (carmustine implant), HERCEPTIN (trastuzumab), HYCAMTIN (topotecan hydrochloride), Hyper-CVAD, ICE, ISTODAX (romidepsin), ICLUSIG (ponatinib hydrochloride), IDAMYCIN (idarubicin), IMBRUVICA (ibrutinib), IFEX (ifosfamide), IFOSFAMIDUM (ifosfamide), INLYTA (axitinib), INTRON A (recombinant interferon alfa-2b), IRESSA (gefitinib), IXEMPRA (ixabepilone), JAKAFI (ruxolitinib phosphate), JEVTANA (cabazitaxel), KADCYLA (ado-trastuzumab emtansine), KEYTRUDA (pembrolizumab), KYPROLIS (carfilzomib), Lomustine, LEUKERAN (chlorambucil), LEUSTATIN (cladribine), LINFOLIZIN (chlorambucil), LIPODOX (doxorubicin hydrochloride liposome), LUPRON (leuprolide acetate), LUPRON DEPOT (leuprolide acetate), LUPRON DEPOT-3 MONTH (leuprolide acetate), LUPRON DEPOT-4 MONTH (leuprolide acetate), LUPRON DEPOT-PED (leuprolide acetate), MATULANE (procarbazine hydrochloride), MOPP, MOZOBIL (plerixafor), MARQIBO (vincristine sulfate liposome), MYLERAN (busulfan), MEGACE (megestrol acetate), MEKINIST (trametinib), METHAZOLASTONE (temozolomide), METHOTREXATE LPF (methotrexate), MEXATE (methotrexate), MEXATE-AQ (methotrexate), MITOXANTRONE HYDROCHLORIDE, MITOZYTREX (mitomycin c), MOZOBIL (plerixafor), MUSTARGEN (mechlorethamine hydrochloride), MUTAMYCIN (mitomycin c), MYLOSAR (azacitidine), NAVELBINE (vinorelbine tartrate), NEOSAR (cyclophosphamide), NEXAVAR (sorafenib tosylate), NOLVADEX (tamoxifen citrate), NOVALDEX (tamoxifen citrate), OEPA, ONTAK (denileukin diftitox), OPPA, OFF, ONCASPAR (Pegaspargase), PAD, PARAPLAT (carboplatin), PARAPLATIN (carboplatin), PEG-INTRON (peginterferon alfa-2b), PEMETREXED DISODIUM, PERJETA (pertuzumab), PLATINOL (cisplatin), PLATINOL-AQ (cisplatin), POMALYST (pomalidomide), prednisone, PROLEUKIN (aldesleukin), PROLIA (denosumab), PROVENGE (sipuleucel-t), PURINETHOL (mercaptopurine), PURIXAN (mercaptopurine), REVLIMID (lenalidomide), R-CHOP, RUBIDOMYCIN (daunorubicin hydrochloride), RITUXAN (rituximab), SPRYCEL (dasatinib), STANFORD V, SYNRIBO (omacetaxine mepesuccinate), STIVARGA (regorafenib), SUTENT (sunitinib malate), SYLATRON (peginterferon alfa-2b), SYLVANT (siltuximab), SYNOVIR (thalidomide), TAC, TAFINLAR (dabrafenib), TARABINE PFS (cytarabine), TARCEVA (erlotinib hydrochloride), TASIGNA (nilotinib), TAXOL (paclitaxel), TAXOTERE (docetaxel), TEMODAR (temozolomide), THALOMID (thalidomide), TOPOSAR (etoposide), TORISEL (temsirolimus), TPF, TREANDA (bendamustine hydrochloride), TRISENOX (arsenic trioxide), TYKERB (lapatinib ditosylate), VAMP, VECTIBIX (panitumumab), VEIP, VELBAN (vinblastine sulfate), VELCADE (bortezomib), VELSAR (vinblastine sulfate), VEPESID (etoposide), VIADUR (leuprolide acetate), VIDAZA (azacitidine), VINCASAR PFS (vincristine sulfate), VOTRIENT (pazopanib hydrochloride), WELLCOVORIN (leucovorin calcium), XALKORI (crizotinib), XELODA (capecitabine), XELOX, XGEVA (denosumab), XOFIGO (radium 223 dichloride), XTANDI (enzalutamide), YERVOY (ipilimumab), ZALTRAP (ziv-aflibercept), ZELBORAF (vemurafenib), ZEVALIN (ibritumomab tiuxetan), ZOLINZA (vorinostat), ZOLADEX (goserelin acetate), ZOMETA (zoledronic acid), ZYDELIG (idelalisib), ZYKADIA (ceritinib), ZYTIGA (abiraterone acetate), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (Velcade)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine, or a combination thereof. In certain embodiments, the additional pharmaceutical agent is a binder or inhibitor of an HMT (e.g., EZH1, EZH2, DOT1). In certain embodiments, the additional pharmaceutical agent is a protein kinase inhibitor (e.g., tyrosine protein kinase inhibitor). In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation.

In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., a proliferative disease, an inflammatory disease, an autoimmune disease, an infectious disease, an allergy, a fibrotic disease, a cardiovascular disease, a graft rejection, graft-versus-host disease, chronic disease, or addiction) in a subject in need thereof. In certain embodiments, the kits are useful for preventing a disease (e.g., a proliferative disease, an inflammatory disease, an autoimmune disease, an infectious disease, an allergy, a fibrotic disease, a cardiovascular disease, a graft rejection, graft-versus-host disease, chronic disease, or addiction) in a subject in need thereof. In certain embodiments, the kits are useful as enhancers and/or modifiers of an immune response (e.g., innate and/or adaptive immune response), and/or adjuvants in a vaccine for a disease, (e.g., a proliferative disease, an inflammatory disease, an autoimmune disease, an infectious disease, an allergy, a fibrotic disease, a cardiovascular disease, a graft rejection, graft-versus-host disease, chronic disease, or addiction) in a subject, biological sample, tissue, or cell, or as stand alone anti-infective or immune response modifying agents.

In certain embodiments, a kit described herein further includes instructions for using the compound or pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., a proliferative disease, an inflammatory disease, an autoimmune disease, an infectious disease, an allergy, a fibrotic disease, a cardiovascular disease, a graft rejection, graft-versus-host disease, chronic disease, or addiction) in a subject in need thereof. In certain embodiments, the kits and instructions provide for preventing a disease (e.g., a proliferative disease, an inflammatory disease, an autoimmune disease, an infectious disease, an allergy, a fibrotic disease, a cardiovascular disease, a graft rejection, graft-versus-host disease, chronic disease, or addiction) in a subject in need thereof. In certain embodiments, the kits and instructions provide for enhancing of an immune response (e.g., innate and/or adaptive immune response) in a subject, biological sample, tissue, or cell. In certain embodiments, the kits and instructions provide for use of the compound as an adjuvant in a vaccine for a disease, (e.g., a proliferative disease, an inflammatory disease, an autoimmune disease, an infectious disease, an allergy, a fibrotic disease, a cardiovascular disease, a graft rejection, graft-versus-host disease, chronic disease, or addiction) in a subject, biological sample, tissue, or cell, or as stand alone anti-infective or immune response modifying agents. A kit described herein may include one or more additional pharmaceutical agents described herein as a separate composition.

### Antigens

The present disclosure also provides compositions comprising an antigen and Compound (1), or a pharmaceutically acceptable salt, solvate, hydrate, polymorph, co-crystal, tautomer, stereoisomer, or isotopically labeled derivative. An “antigen” refers to an entity that is bound by an antibody or receptor, or an entity that induces the production of the antibody. In some embodiments, an antigen increases the production of antibodies that specifically bind the antigen. In some embodiments, an antigen comprises a protein or polypeptide. Such protein or peptide are referred to herein as “immunogenic polypeptide.” In some embodiments, the term “antigen” encompasses nucleic acids (e.g., DNA or RNA molecules) that encode immunogenic polypeptides. In some embodiments, the antigen is from a microbial pathogen. For example, the antigen may comprise parts (coats, capsules, cell walls, flagella, fimbriae, and toxins) of bacteria, viruses, fungi, and other microorganisms. In some embodiments, the antigen is a cancer-specific antigen. In some embodiments, the antigen is a psychoactive substance or its hapten derivative (e.g., an opioid-specific antigen). In some embodiments, the antigen is a hapten.

In some embodiments, a protein or polypeptide antigen is a wild type protein or polypeptide. In some embodiments, a protein or polypeptide antigen is a polypeptide variant to a wild type protein or polypeptide. The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. In some embodiments, polypeptide variants possess at least 50% identity to a native or reference sequence. In some embodiments, variants share at least 70%, at least 80%, at least 90%, at least 95%, or at least 99% identity with a native or reference sequence.

In some embodiments, a polypeptide variant comprises substitutions, insertions, deletions. In some embodiments, a polypeptide variant encompasses covalent variants and derivatives. The term “derivative” is used synonymously with the term “variant” but generally refers to a molecule that has been modified and/or changed in any way relative to a reference molecule or starting molecule.

In some embodiments, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble or linked to a solid support.

In some embodiments, the polypeptide variant comprises at least one amino acid residue in a native or starting sequence removed and a different amino acid inserted in its place at the same position. Substitutions may be single, where only one amino acid in the molecule has been substituted, or they may be multiple, where two or more amino acids have been substituted in the same molecule. In some embodiments, the antigen is a polypeptide that includes 2, 3, 4, 5, 6, 7, 8, 9, 10, or more substitutions compared to a reference protein.

In some embodiments, the substitution is a conservative amino acid substitution. The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

In some embodiments, protein fragments, functional protein domains, and homologous proteins are used as antigens in accordance with the present disclosure. For example, an antigen may comprise any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length. In another example, any protein that includes a stretch of 20, 30, 40, 50, or 100 amino acids which are 40%, 50%, 60%, 70%, 80%, 90%, 95%, or 100% identical to a reference protein (e.g., a protein from a microbial pathogen) herein can be utilized in accordance with the disclosure.

In some embodiments, the antigen comprises more than one immunogenic protein or polypeptide (e.g., 2, 3, 4, 5, 6, 7, 8, 9, 10, or more). In some embodiments, the more than one immunogenic protein or polypeptide are derived from one protein (e.g., different fragments or one protein). In some embodiments, the more than one immunogenic protein or polypeptide are derived from multiple proteins (e.g., from 2, 3, 4, 5, 6, 7, 8, 9, 10, or more proteins).

In some embodiments, the antigen comprises a nucleic acid encoding an immunogenic protein or polypeptide. In some embodiments, the antigen comprises an immunogenic protein or polypeptide and a nucleic acid encoding the immunogenic protein or polypeptide. The term “nucleic acid” or “polynucleotide,” in its broadest sense, includes any compound and/or substance that comprises a polymer of nucleotides. Nucleic acids encoding immunogenic proteins or polypeptides typically comprise an open reading frame (ORF), and one or more regulatory sequences. Nucleic acids (also referred to as polynucleotides) may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or chimeras or combinations thereof.

In some embodiments, the nucleic acid encoding the immunogenic polypeptide is a DNA (e.g., an expression vector for an immunogenic protein or polypeptide). In some embodiments, the nucleic acid encoding the immunogenic polypeptide is an RNA (e.g., a messenger RNA). A “messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ, or ex vivo. The basic components of an mRNA molecule typically include at least one coding region, a 5′ untranslated region (UTR), a 3′ UTR, a 5′ cap and a poly-A tail.

In some embodiments, the coding region of the nucleic acid (e.g., DNA or RNA) encoding an immunogenic polypeptide is codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence (e.g., a naturally-occurring or wild-type mRNA sequence encoding an immunogenic protein or polypeptide). In some embodiments, a codon optimized sequence shares less than 90% sequence identity to a naturally-occurring or wild-type sequence (e.g., a naturally-occurring or wild-type mRNA sequence encoding an immunogenic protein or polypeptide). In some embodiments, a codon optimized sequence shares less than 85% sequence identity to a naturally-occurring or wild-type sequence (e.g., a naturally-occurring or wild-type mRNA sequence encoding an immunogenic protein or polypeptide). In some embodiments, a codon optimized sequence shares less than 80% sequence identity to a naturally-occurring or wild-type sequence (e.g., a naturally-occurring or wild-type mRNA sequence encoding an immunogenic protein or polypeptide). In some embodiments, a codon optimized sequence shares less than 75% sequence identity to a naturally-occurring or wild-type sequence (e.g., a naturally-occurring or wild-type mRNA sequence encoding an immunogenic protein or polypeptide).

In some embodiments, the nucleic acid encoding an immunogenic protein or polypeptide comprises one or more chemical modifications. The terms “chemical modification” and “chemically modified” refer to modification with respect to adenosine (A), guanosine (G), uridine (U), thymidine (T) or cytidine (C) ribonucleosides or deoxyribnucleosides in at least one of their position, pattern, percent or population.

In some embodiments, the nucleic acids (e.g., DNA or RNA) comprise various (more than one) different modifications. In some embodiments, a particular region of a nucleic acid (e.g., DNA or RNA) contains one, two or more (optionally different) nucleoside or nucleotide modifications. In some embodiments, a modified nucleic acid (e.g., DNA or RNA), introduced to a cell or organism, exhibits reduced degradation in the cell or organism, respectively, relative to an unmodified nucleic acid. In some embodiments, a modified nucleic acid (e.g., DNA or RNA), introduced into a cell or organism, may exhibit reduced immunogenicity in the cell or organism, respectively (e.g., a reduced innate response).

Modified nucleic acid (e.g., DNA or RNA) may comprise modifications that are naturally-occurring, non-naturally-occurring or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone). Modified nucleic acid (e.g., DNA or RNA), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

In some embodiments, a chemically modified nucleic acid comprises one or more modified nucleosides. A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides.

In some embodiments, a modified nucleobase is a modified uridine. Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine.

In some embodiments, a modified nucleobase is a modified uridine. In some embodiments, a modified nucleobase is a modified cytosine nucleosides having a modified uridine include 5-cyano uridine, and 4′-thiouridine.

In some embodiments, a modified nucleobase is a modified adenine. Exemplary nucleobases and nucleosides having a modified adenine include 7-deaza-adenine, 1-methyl-adenosine (m1A), 2-methyl-adenine (m2A), and N6-methyl-adenosine (m6A).

In some embodiments, a modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (mlG), 8-oxo-guanosine, 7-methyl-8-oxo-guanosine.

In some embodiments, the antigen of the present disclosure is from a microbial pathogen, e.g., from a bacterium, mycobacterium, fungus, a virus, parasite, or prion. For example, the antigen may comprise a protein or polypeptide, or a nucleic acid encoding the protein or polypeptide from the microbial pathogen. In some embodiments, the antigen may comprise a microbial pathogen (e.g., a bacterial cell, a viral particle, or a fungus cell). In some embodiments, the microbial pathogen cell is live or killed. In some embodiments, the live microbial pathogen is attenuated with respect to its pathogenicity. An attenuated microbial pathogen may elicit immune response but does not cause the disease that a wild-type microbial pathogen would cause.

Exemplary, non-limiting bacterial taxa, species, and strains, suitable for use in some embodiments of this disclosure include: Escherichia spp., Enterobacter spp. (e.g., Enterobacter cloacae), Salmonella spp. (e.g., Salmonella enteritidis, Salmonella typhi), Shigella spp., Pseudomonas spp. (e.g., Pseudomonas aeruginosa, Pseudomonas pachastrellae, Pseudomonas stutzeri), Moraxella spp. (e.g., Moraxella catarrhalis), Neisseria spp. (e.g., Neisseria gonorrhoeae, Neisseria meningitidis), Helicobacter spp., (e.g., Helicobacter pylori) Stenotrophomonas spp., Vibrio spp. (e.g., Vibrio cholerae), Legionella spp. (Legionella pneumophila), Hemophilus spp. (e.g., Hemophilus influenzae), Klebsiella spp. (e.g., Klebsiella pneumoniae), Proteus spp. (e.g., Proteus mirabilis), Serratia spp. (Serratia marcescens), Streptococcus spp., Staphylococcus spp., Corynebacterium spp., Listeria spp., Bacillus spp. (e.g., Bacillus anthracis) Bordetella spp. (e.g., Bordetella pertussis); Borrelia spp. (e.g., Borrelia burgdorferi); Brucella spp. (e.g., Brucella abortus, Brucella canis, Brucella melitensis, Brucella suis); Campylobacter spp. (e.g., Campylobacter jejuni); Chlamydia spp. and Chlamydophila spp. (e.g., Chlamydia pneumoniae, Chlamydia trachomatis, Chlamydophila psittaci); Clostridium spp. (e.g., Clostridium botulinum, Clostridium difficile, Clostridium perfringens, Clostridium tetani); Corynebacterium spp. (e.g., Corynebacterium diphtheriae); Enterococcus spp. (e.g., Enterococcus faecalis, Enterococcus faecium); Escherichia spp. (e.g., Escherichia coli, Enterotoxic E. coli, enteropathogenic E. coli; E. coli O157:H7); Francisella spp. (e.g., Francisella tularensis); Haemophilus spp. (e.g., Haemophilus influenzae); Helicobacter spp. (e.g., Helicobacter pylori); Legionella spp. (e.g., Legionella pneumophila); Leptospira spp. (e.g., Leptospira interrogans); Listeria spp. (e.g., Listeria monocytogenes); Mycobacterium spp. (e.g., Mycobacterium leprae, Mycobacterium tuberculosis, Mycobacterium ulcerans); Mycoplasma spp. (e.g., Mycoplasma pneumoniae); Neisseria spp. (e.g., Neisseria gonorrhoeae, Neisseria meningitidis); Pseudomonas spp. (e.g., Pseudomonas aeruginosa); Rickettsia spp. (e.g., Rickettsia rickettsii); Salmonella spp. (e.g., Salmonella typhi, Salmonella typhimurium); Shigella spp. (e.g., Shigella sonnei); Staphylococcus spp. (e.g., Staphylococcus aureus, Staphylococcus epidermidis, Staphylococcus saprophyticus); Streptococcus spp. (e.g., Streptococcus agalactiae, Streptococcus pneumoniae, Streptococcus pyogenes); Treponema spp. (e.g., Treponema pallidum); Pseudodiomarina spp. (e.g., P. maritima); Marinobacter spp. (e.g., Marinobacter hydrocarbonoclasticus, Marinobacter vinifirmus) Alcanivorax spp. (e.g., alcanivorax dieselolei); Acetinobacter spp. (e.g., A. venetianus); Halomonas spp. (e.g., H. shengliensis); Labrenzia spp.; Microbulifer spp. (e.g., M. schleiferi); Shewanella spp. (e.g., S. algae); Vibrio spp. (e.g., Vibrio cholerae, Vibrio alginolyticus, Vibrio hepatarius); and Yersinia spp. (e.g., Yersinia pestis).

In some embodiments, the bacterium is Bacillus anthracis (causing anthrax), Bordetella pertussis (causing whooping cough), Corynebacterium diphtheriae (causing diphtheria), Clostridium tetani (causing tetanus), Haemophilus influenzae type b, pneumococcus (causing pneumococcal infections), Staphylococci spp. (including Group A or B streptococci), Mycobacterium tuberculosis, Neiserria meningitidis (causing meningococcal disease), Salmonella typhi (causing typhoid), Vibrio cholerae (causing Cholera), or Yersinia pestis (causing plague).

In some embodiments, the antigen is derived from a Gram-negative bacterium. In some embodiments, the antigen is a lipopolysaccharide endotoxin (LPS) from a Gram-negative bacterium. Non-limiting examples of Gram-negative bacterial species include: Neisseria species including Neisseria gonorrhoeae and Neisseria meningitidis, Branhamella species including Branhamella catarrhalis, Escherichia species including Escherichia coli, Enterobacter species, Proteus species including Proteus mirabilis, Pseudomonas species including Pseudomonas aeruginosa, Pseudomonas mallei, and Pseudomonas pseudomallei, Klebsiella species including Klebsiella pneumoniae, Salmonella species, Shigella species, Serratia species, Acinetobacter species; Haemophilus s pecies including Haemophilus influenzae and Haemophilus ducreyi; Brucella species, Yersinia species including Yersinia pestis and Yersinia enterocolitica, Francisella species including Francisella tularensis, Pasteurella species including Pasteurella multocida, Vibrio cholerae, Flavobacterium species, meningosepticum, Campylobacter species including Campylobacter jejuni, Bacteroides species (oral, pharyngeal) including Bacteroides fragilis, Fusobacterium species including Fusobacterium nucleatum, Calymmatobacterium granulomatis, Streptobacillus species including Streptobacillus moniliformis, Legionella species including Legionella pneumophila.

In some embodiments, the antigen is derived from a Gram-positive bacterium. Exemplary Gram-positive bacteria include, but are not limited to, Staphylococcus spp., Streptococcus spp., Micrococcus spp., Peptococcus spp., Peptostreptococcus spp., Enterococcus spp., Bacillus spp., Clostridium spp., Lactobacillus spp., Listeria spp. Erysipelothrix spp., Propionibacterium spp., Eubacterium spp., Corynebacterium spp., Capnocytophaga spp., Bifidobacterium spp., and Gardnerella spp. In some embodiments, the Gram-positive bacteria is a bacteria of the phylum Firmicutes. In some embodiments, the Gram-positive bacteria is Streptococcus.

Other types of bacteria include acid-fast bacilli, spirochetes, and actinomycetes. Examples of acid-fast bacilli include Mycobacterium species including Mycobacterium tuberculosis and Mycobacterium leprae. Examples of spirochetes include Treponema species including Treponema pallidum, Treponena pertenue, Borrelia species including Borrelia burgdorferi (Lyme disease), and Borrelia recurrentis, and Leptospira species. Examples of actinomycetes include: Actinomyces species including Actinomyces israelii, and Nocardia species including Nocardia asteroides.

Examples of viruses include but are not limited to: Retroviruses, human immunodeficiency viruses including HIV-1, HDTV-III, LAVE, HTLV-III/LAV, HIV-III, HIV-LP, Cytomegaloviruses (CMV), Picornaviruses, polio viruses, hepatitis A virus, enteroviruses, human Coxsackie viruses, rhinoviruses, echoviruses, Calciviruses, Togaviruses, equine encephalitis viruses, rubella viruses, Flaviruses, dengue viruses, encephalitis viruses, yellow fever viruses, Coronaviruses (e.g., SARS-CoV-2), Rhabdoviruses, vesicular stomatitis viruses, rabies viruses, Filoviruses, ebola virus, Paramyxoviruses, parainfluenza viruses, mumps virus, measles virus, respiratory syncytial virus (RSV), Orthomyxoviruses, influenza viruses, Bungaviruses, Hantaan viruses, phleboviruses and Nairo viruses, Arena viruses, hemorrhagic fever viruses, reoviruses, orbiviruses, rotaviruses, Birnaviruses, Hepadnaviruses, Hepatitis B virus, parvoviruses, Papovaviridae, papilloma viruses, polyoma viruses, Adenoviruses, herpes viruses (e.g., herpes simplex virus 1, herpes simplex virus 2, human herpesvirus 6, human herpesvirus 8), varicella zoster virus, Poxviruses, variola viruses, vaccinia viruses, Irido viruses, African swine fever virus, delta hepatitis virus, non-A, non-B hepatitis virus, Hepatitis C, Norwalk viruses, astroviruses, and unclassified viruses. In some embodiments, the virus is adenovirus, enterovirus such as polio virus, Ebola virus, cytomegalovirus and varicella-zoster (chickenpox and shingles), measles, mumps, rubella, hepatitis-A, -B, or -C, human papilloma virus, Influenza virus, parainfluenza virus, rabies, Japanese encephalitis, rotavirus, human immunodeficiency virus (HIV), respiratory syncytial virus (RSV), smallpox, monkeypox, yellow fever, or Zika Virus. In some embodiments, the virus is influenza or a coronavirus (e.g., SARS-CoV-2).

In some embodiments, the antigen comprises a viral protein and/or a nucleic acid encoding a viral protein (e.g., a viral structural or non-structural protein). In some embodiments, the antigen comprises a nucleic acid encoding the viral genome. In some embodiments, the viral genome is modified to produce a modified virus that is attenuated.

Examples of fungus include, but are not limited to: Cryptococcus species including Cryptococcus neoformans, Histoplasma species including Histoplasma capsulatum, Coccidioides species including Coccidiodes immitis, Paracoccidioides species including Paracoccidioides brasiliensis, Blastomyces species including Blastomyces dermatitidis, Chlamydia species including Chlamydia trachomatis, Candida species including Candida albicans and Candida auris, Sporothrix species including Sporothrix schenckii, Aspergillus species, and fungi of mucormycosis. In some embodiments, the fungus is Candida spp., Aspergillus spp., Cryptococcus spp., Mucormycete, Blastomyces dermatitidis (causing blastomycosis), or endemic mycosis causing fungus such as Histoplasma capsulatum (causing histoplasmosis), or Sporothrix schenckii (causing sporotrichosis).

Other infectious organisms include, without limitation: parasites. Parasites include Plasmodium species, such as Plasmodium species including Plasmodium falciparum, Plasmodium malariae, Plasmodium ovae, and Plasmodium vivax and Toxoplasma gondii. Blood-borne and/or tissues parasites include Plasmodium species, Babesia species including Babesia microti and Babesia divergens, Leishmania species including Leishmania tropica, Leishmnania species, Leishmania braziliensis, Leishmania donovani, Trypanosoma species including Trypanosoma gambiense, Trypanosoma rhodesiense (African sleeping sickness), and Trypanosoma cruzi (Chagas' disease). In some embodiments, the parasite is Plasmodium spp., Leishmania, or a helminth.

Other medically relevant microorganisms have been described extensively in the literature, e.g., see C. C. A Thomas, Medical Microbiology, Bailliere Tindall, Great Britain 1983, incorporated herein by reference.

In some embodiments, the antigen of the present disclosure comprises a cancer-specific antigen and/or a nucleic acid encoding such. A “cancer-specific antigen” refers to a protein that is specifically expressed or upregulated in a cancer cell, as compared to non-cancerous cells of the same origin. A cancer-specific antigen, or epitopes derived therefrom, can be recognized by the immune system to induce an immune response against the cancer. Classes of proteins that may be cancer-specific antigen include, without limitation: enzymes, receptors, and transcription factors.

A large number of proteins that specifically express in cancer cells or are upregulated in cancer cells have been identified (Hassane et al., Holland-Frei Cancer Medicine. 6th edition, incorporated herein by reference). The known tumor specific antigens are classified into different classes: cancer-testis antigens (e.g., MAGE family members or NY-ESO-1), differentiation antigens (e.g., tyrosinase and Melan-A/MART-1 for melanoma, and PSA for prostate cancer), overexpressed cancer-specific antigens (e.g., Her-2/neu, Survivin, Telomerase and WT1), cancer-specific antigens arising from mutations of normal genes (e.g., mutated β-catenin or CDK4), cancer-specific antigens arising from abnormal post-translational modifications (e.g., altered glycosylation patterns) that lead to novel epitopes in tumors (e.g., MUC1), and oncoviral proteins (e.g., human papilloma type 16 virus proteins, E6 and E7). In some embodiments, the tumor-specific antigen is expressed in a broad range of different types of cancers. In some embodiments, the tumor-specific antigen is expressed only in one or a few types of cancers.

In some embodiments, the antigen comprises a fragment or an epitope derived from a cancer-specific antigen and/or a nucleic acid encoding such. For example, the fragment or an epitope derived from a cancer-specific antigen may be 5-40 amino acids long. In some embodiments, the fragment or an epitope derived from a cancer-specific antigen is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids long.

In some embodiments, the fragment or epitope derived from a cancer-specific antigen is a heteroclitic epitope. A “heteroclitic epitope” refers to an altered version of an endogenous peptide sequence (i.e., an analog) from a cancer-specific antigen engineered to elicit potent immune reactions. Heteroclitic epitopes have increased stimulatory capacity or potency for a specific T cell, as measured by increased responses to a given dose, or by a requirement of lesser amounts to achieve the same response and therefore provide benefit as vaccine components since these epitopes induce T cell responses stronger than those induced by the native epitope.

In some embodiments, the heteroclitic epitope comprises modifications, e.g., amino acid substitutions, as compared to the native sequence in the cancer-specific antigen. In some embodiments, the heteroclitic epitope comprises more than one amino acid substitutions (e.g., 2, 3, 4, 5, or more) compared to the native sequence of the cancer-specific antigen it is derived from. In some embodiments, a heteroclitic epitope is at least 60%, at least 70%, at least 80%, at least 90%, at least 98%, or at least 99% identical to the native sequence that it is derived from. In some embodiments, a heteroclitic epitope is 60%, 65%, 70%, 75%, 80%, 85%, 90%, 95%, 98%, or 99% identical to the native sequence that it is derived from.

In some embodiments, a heteroclitic epitope is more immunogenic than a peptide of its native sequence. For example, a heteroclitic epitope may be at least 30% more immunogenic (i.e., induces a stronger immune response) than its corresponding native peptide. In some embodiments, a heteroclitic epitope may be at least 30%, at least 40%, at least 50%, at least 60%, at least 70%, at least 80%, at least 90%, at least 100%, at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 100-fold, or more immunogenic than its corresponding native peptide.

In some embodiments, the fragment or epitope derived from a cancer-specific antigen is a cryptic epitope. A “cryptic epitope” refers to an epitope derived from a cancer-specific antigen that does not necessarily undergo antigen processing/presentation and are ‘hidden’ from immune recognition. Cryptic epitopes usually appear in very low concentration on APC and do not delete auto-reactive T cells. Cryptic epitopes are not presented for recognition by T cells unless they are produced in unusually large concentrations or unless they are freed from the configuration of their native antigen. Cryptic epitopes derived from cancer-specific antigens may be used to break the tolerance of T cells to the tumor and induce potent immune response against the tumor. Such principles have been described in Pardoll, et al., PNAS, Vol. 96, pp. 5340-5342 (1999), the entire contents of which are incorporated herein by reference.

In some embodiments, the cryptic epitope is generated from translation of a non-coding region of the cancer-specific antigen gene or translation of a different reading frame of a coding region of the cancer-specific antigen. A cryptic epitope may be more immunogenic (i.e., induces a stronger immune response) than any native peptide derived from the cancer-specific antigen. For example, a cryptic epitope may be at least 30% more immunogenic than any native peptide derived from the cancer-specific antigen. In some embodiments, a cryptic epitope is at least 30%, at least 40%, at least 50%, at least 60%, at least 70%, at least 80%, at least 90%, at least 100%, at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 100-fold, or more immunogenic than any native peptide derived from the cancer-specific antigen. One skilled in the art is familiar with how to assess the immune response induced by an antigen, e.g., measuring antibody titers.

In some embodiments, the cancer-specific antigen is a neoantigen. A “neoantigen” refers to an antigen generated via random somatic mutations occurring in cancer cells and are thus specific to the lineage of cancer cells it is derived from. Neoantigens are regarded in the art to be responsible for the immunogenicity of tumors ((Srivastava et al., 1993, Duan et al., 2009; van der Bruggen et al., 2013, incorporated herein by reference), and mathematic modeling has predicted the existence of tens to hundreds of neoepitopes (epitopes derived from neoantigens) in individual human tumors (Srivastava 2009, incorporated herein by reference). The recent revolution in high-throughput DNA sequencing and accompanying bioinformatics approaches has finally made it possible to accurately identify the individually specific neoepitopes in individual cancers.

In some embodiments, the antigen described herein is an antigen designed to provide broad heterologous protection against a range of pathogens. Heterologous immunity refers to the phenomenon whereby a history of an immune response against a stimulus or pathogen can provide a level of immunity to a second unrelated stimulus or pathogen (e.g., as described in Chen et al., Virology 2015 482: 89-97, incorporated herein by reference). For example, an antigen that induces cross-reactive memory CD8+ T cells against multiple unrelated viruses such as influenza A and Epstein-Barr Virus (EBV), as described in Watkin et al., J Allerg Clin Immunol 2017 October; 140(4) 1206-1210, incorporated herein by reference. In some embodiments, Compound (1) induces and/or enhances the heterologous protection.

Polypeptide or polynucleotide molecules of the present disclosure may share a certain degree of sequence similarity or identity with reference molecules (e.g., reference polypeptides or reference polynucleotides), for example, wild-type molecules. The term “identity” as known in the art and described below, refers to a relationship between the sequences of two or more polypeptides or polynucleotides, as determined by comparing the sequences. In the art, identity also means the degree of sequence relatedness between them as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197.) A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453.). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm. Other tools are described herein, specifically in the definition of “identity” below.

As used herein, the term “homology” refers to the overall relatedness between polymeric molecules, e.g., between nucleic acid molecules (e.g., DNA molecules and/or RNA molecules) and/or between polypeptide molecules. Polymeric molecules (e.g., nucleic acid molecules (e.g. DNA molecules and/or RNA molecules) and/or polypeptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). Two polynucleotide sequences are considered homologous if the polypeptides they encode are at least 50%, 60%, 70%, 80%, 90%, 95%, or even 99% for at least one stretch of at least 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered homologous if the proteins are at least 50%, 60%, 70%, 80%, or 90% identical for at least one stretch of at least 20 amino acids.

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” are genes (or proteins) related by duplication within a genome. Orthologs retain the same function in the course of evolution, whereas paralogs evolve new functions, even if these are related to the original one. The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In some embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, considering the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

### Compositions and Vaccines

As described herein, the present disclosure provides compositions comprising an antigen and Compound (1), or a pharmaceutically acceptable salt, solvate, hydrate, polymorph, co-crystal, tautomer, stereoisomer, or isotopically labeled derivative thereof. In some embodiments, Compound (1) is conjugated to the antigen. In some embodiments, Compound (1) is not conjugated to the antigen.

Methods of conjugating a compound to another molecule (e.g., a protein or a nucleic acid) is known to those skilled in the art. For example, in some embodiments, conjugation may be achieved via reactive chemical groups by incorporating one of a pair of reactive chemical groups that react with each other to each of the two molecules to be conjugated. A “reactive chemical group” or “functional chemical group” refers to specific groups (moieties) of atoms or bonds within molecules that are responsible for the characteristic chemical reactions of those molecules. These terms are used interchangeably herein. One example of such reactive group is a “click chemistry handle.” Click chemistry is a chemical approach introduced by Sharpless in 2001 and describes chemistry tailored to generate substances quickly and reliably by joining small units together. See, e.g., Kolb, Finn and Sharpless Angewandte Chemie International Edition (2001) 40: 2004-2021; Evans, Australian Journal of Chemistry (2007) 60: 384-395). Exemplary coupling reactions (some of which may be classified as “Click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgon cycloaddition; thiol-yne addition; imine formation; and Michael additions (e.g., maleimide addition). Non-limiting examples of a click chemistry handle include an azide handle, an alkyne handle, or an aziridine handle. Azide is the anion with the formula N₃—. It is the conjugate base of hydrazoic acid (HN₃). N₃— is a linear anion that is isoelectronic with CO₂, NCO—, N₂O, NO₂⁺ and NCF. Azide can be described by several resonance structures, an important one being —N═N⁺═N⁻. An alkyne is an unsaturated hydrocarbon containing at least one carbon-carbon triple bond. The simplest acyclic alkynes with only one triple bond and no other functional groups form a homologous series with the general chemical formula CₙH₂ₙ₋₂. Alkynes are traditionally known as acetylenes, although the name acetylene also refers specifically to C₂H₂, known formally as ethyne using IUPAC nomenclature. Like other hydrocarbons, alkynes are generally hydrophobic but tend to be more reactive. Aziridines are organic compounds containing the aziridine functional group, a three-membered heterocycle with one amine group (—NH—) and two methylene bridges (—CH₂—). The parent compound is aziridine (or ethylene imine), with molecular formula C₂H₅N.

Other non-limiting, exemplary reactive groups include: acetals, ketals, hemiacetals, and hemiketals, carboxylic acids, strong non-oxidizing acids, strong oxidizing acids, weak acids, acrylates and acrylic acids, acyl halides, sulfonyl halides, chloroformates, alcohols and polyols, aldehydes, alkynes with or without acetylenic hydrogen amides and imides, amines, aromatic, amines, phosphines, pyridines, anhydrides, aryl halides, azo, diazo, azido, hydrazine, and azide compounds, strong bases, weak bases, carbamates, carbonate salts, chlorosilanes, conjugated dienes, cyanides, inorganic, diazonium salts, epoxides, esters, sulfate esters, phosphate esters, thiophosphate esters borate esters, ethers, soluble fluoride salts, fluorinated organic compounds, halogenated organic compounds, halogenating agents, aliphatic saturated hydrocarbons, aliphatic unsaturated hydrocarbons, hydrocarbons, aromatic, insufficient information for classification, isocyanates and isothiocyanates, ketones, metal hydrides, metal alkyls, metal aryls, and silanes, alkali metals, nitrate and nitrite compounds, inorganic, nitrides, phosphides, carbides, and silicides, nitriles, nitro, nitroso, nitrate, nitrite compounds, organic, non-redox-active inorganic compounds, organometallics, oximes, peroxides, organic, phenolic salts, phenols and cresols, polymerizable compounds, quaternary ammonium and phosphonium salts, strong reducing agents, weak reducing agents, acidic salts, basic salts, siloxanes, inorganic sulfides, organic sulfides, sulfite and thiosulfate salts, sulfonates, phosphonates, organic thiophosphonates, thiocarbamate esters and salts, and dithiocarbamate esters and salts. In some embodiments, the reactive group is a carboxylic acid group.

Compositions comprising an antigen and Compound (1) described herein are immunogenic. Being “immunogenic” means that the composition elicits immune response when administered to a subject (e.g., a mammalian subject such as a human). As used herein, an “immune response” refers to a response by a cell of the immune system, such as an antigen-presenting cell, dendritic cell, monocyte, macrophage, NKT cell, NK cell, basophil, eosinophil, or neutrophil, B cell, T cell (CD4 or CD8), regulatory T cell, antigen-presenting cell, dendritic cell, monocyte, macrophage, NKT cell, NK cell, basophil, eosinophil, or neutrophil, to a stimulus (e.g., to an antigen or an adjuvant).

In some embodiments, the immune response elicited by the composition described herein is specific for a particular antigen (an “antigen-specific response” or “adaptive immune response”) and refers to a response by a CD4⁺ T cell, CD8⁺ T cell, or B cell via their antigen-specific receptor. In some embodiments, an immune response is a T cell response, such as a CD4⁺ response or a CD8⁺ response. Such responses by these cells can include, for example, cytotoxicity, proliferation, cytokine or chemokine production, trafficking, or phagocytosis, and can be dependent on the nature of the immune cell undergoing the response.

In some embodiments, an antigen-specific immune response includes both a humoral and/or a cell-mediated immune response to the antigen. A “humoral immune response” is an antibody-mediated immune response and involves the induction and generation of antibodies that recognize and bind with some affinity for the antigen in the immunogenic composition of the invention, while a “cell-mediated immune response” is one mediated by T-cells and/or other white blood cells. A “cell-mediated immune response” is elicited by the presentation of antigenic epitopes in association with Class I or Class II molecules of the major histocompatibility complex (MHC), CD1 or other non-classical MHC-like molecules. This activates antigen-specific CD4⁺ T helper cells or CD8⁺ cytotoxic lymphocyte cells (“CTLs”). CTLs have specificity for peptide antigens that are presented in association with proteins encoded by classical or non-classical MHCs and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide or other antigens in association with classical or non-classical MHC molecules on their surface. A “cell-mediated immune response” also refers to the production of cytokines, chemokines, and other such molecules produced by activated T-cells and/or other white blood cells, including those derived from CD4⁺ and CD8⁺ T-cells. The ability of a particular antigen or composition to stimulate a cell-mediated immunological response may be determined by various assays, such as by lymphoproliferation (lymphocyte activation) assays, CTL cytotoxic cell assays, by assaying for T-lymphocytes specific for the antigen in a sensitized subject, or by measurement of cytokine production by T cells in response to re-stimulation with antigen. Such assays are well known in the art. See, e.g., Erickson et al. (1993) J. Immunol. 151:4189-4199; and Doe et al. (1994) Eur. J. Immunol. 24:2369-2376.

In some embodiments, the immune response elicited by a composition described herein is an innate immune response. An “innate immune response” refers to the response by the innate immune system. The innate immune system uses a set of germline-encoded receptors (“pattern recognition receptor” or “PRR”) for the recognition of conserved molecular patterns present in microorganisms. These molecular patterns occur in certain constituents of microorganisms including lipopolysaccharides, peptidoglycans, lipoteichoic acids, phosphatidyl cholines, bacteria-specific proteins, including lipoproteins, bacterial DNAs, viral single and double-stranded RNAs, unmethylated CpG-DNAs, mannans, and a variety of other bacterial and fungal cell wall components. Such molecular patterns can also occur in other molecules such as plant alkaloids. These targets of innate immune recognition are called Pathogen Associated Molecular Patterns (PAMPs) since they are produced by microorganisms and not by the infected host organism. In some embodiments, the innate immune response elicited by the composition described herein confers heterologous (“non-specific”) immunity to a broad range of pathogenic microbes by enhancing innate immune responses to subsequent stimuli, a phenomenon known as “trained immunity”, a form of innate memory, e.g., as described in Netea et al. (Trained Immunity: An Ancient Way of Remembering. Cell Host Microbe. 2017 Mar. 8; 21(3):297-300, incorporated herein by reference).

The receptors of the innate immune system that recognize PAMPs are called Pattern Recognition Receptors (PRRs). (Janeway et al. (1989) Cold Spring Harb. Symp. Quant. Biol. 54: 1-13; Medzhitov et al. (1997) Curr. Opin. Immunol. 94: 4-9, incorporated herein by reference). PRRs vary in structure and belong to several different protein families. Some of these receptors recognize PAMPs directly (e.g., CD14, DEC205, collectins), while others (e.g., complement receptors) recognize the products generated by PAMP recognition. Members of these receptor families can, generally, be divided into three types: 1) humoral receptors circulating in the plasma; 2) endocytic receptors expressed on immune-cell surfaces, and 3) signaling receptors that can be expressed either on the cell surface or intracellularly. (Medzhitov et al. (1997) Curr. Opin. Immunol. 94: 4-9; Fearon et al. (1996) Science 272: 50-3, incorporated herein by reference). Non-limiting examples of PRRs include: toll-like receptors (e.g., TLR2), NOD1/2, RIG-1/MDA-5, C-type lectins, and STING.

Cellular PRRs are expressed on effector cells of the innate immune system, including cells that function as professional antigen-presenting cells (APC) in adaptive immunity. Such effector cells include, but are not limited to, macrophages, dendritic cells, B lymphocytes and surface epithelia. This expression profile allows PRRs to directly induce innate effect or mechanisms, and also to alert the host organism to the presence of infectious agents by inducing the expression of a set of endogenous signals, such as inflammatory cytokines and chemokines, including, without limitation: chemokines, interferons, interleukins, lymphokines, and tumour necrosis factors. This latter function allows efficient mobilization of effector forces to combat the invaders.

In some embodiments, the composition comprising an antigen and Compound (1) is a vaccine composition. A “vaccine composition” is a composition that activates or enhances a subject's immune response to an antigen after the vaccine is administered to the subject. In some embodiments, a vaccine stimulates the subject's immune system to recognize the antigen as foreign and enhances the subject's immune response if the subject is later exposed to the pathogen, whether attenuated, inactivated, killed, or not. Vaccines may be prophylactic, for example, preventing or ameliorating a detrimental effect of a future exposure to a pathogen, or therapeutic, for example, activating the subject's immune response to a pathogen after the subject has been exposed to the pathogen. In some embodiments, a vaccine composition is used to protect or treat an organism against a disease (e.g., an infectious disease or cancer). In some embodiments, the vaccine is a subunit vaccine (e.g., a recombinant subunit vaccine), an attenuated vaccine (e.g., containing an attenuated pathogen such as a bacterial cell or a viral genome), a live vaccine (e.g., containing a live attenuated pathogen such as a bacterium or virus), a conjugated vaccine (e.g., a vaccine containing an antigen that is not very immunogenic covalently attached to an antigen that is more immunogenic) or an mRNA vaccine. One non-limiting example of a conjugated vaccine comprises a LPS attached to a strong protein antigen. The terms “vaccine composition” and “vaccine” are used interchangeably herein.

Vaccines that contain cancer-specific antigens are termed herein as “cancer vaccine.” Cancer vaccines induce cancer-specific immune responses against a cancer or a cancer-specific antigen. Such immunoresponse is effective in inhibiting cancer growth and/or preventing reoccurrence of tumor. Cancer vaccines may be used for cancer immunotherapy, which is a type of cancer treatment designed to boost the body's natural defenses to fight the cancer. It uses substances either made by the body or in a laboratory to improve or restore immune system function.

In some embodiments, Compound (1) is used as an adjuvant in a vaccine composition (e.g., to enhance an immune response in a subject). Compound (1) alone may induce cytokine (e.g., proinflammatory cytokines such as TNF, IL-12, IL-6, or IL-10) and/or chemokine (e.g., CXCL8) production by human peripheral blood mononuclear cells (PBMCs) in vitro and enhance antigen-specific immune response against influenza hemagglutinin antigen in vivo.

In some embodiments, the vaccine composition described herein comprises two or more adjuvants (also referred to as an “adjuvant system”). The adjuvant system comprises Compound (1) and one or more other adjuvants described herein. In some embodiments, the vaccine composition described herein further comprises a second adjuvant in addition to Compound (1) (as the first adjuvant). Any known adjuvants may be used as the second adjuvant in the composition described herein. In some embodiments, Compound (1) and/or the second adjuvant enhance antigen-presenting cell activity. In some embodiments, Compound (1) and/or the second adjuvant activates B cell immunity.

In some embodiments, the second adjuvant is an agonist of Pattern Recognition Receptors (PRRs) such as Toll-like receptors (TLRs), NOD-like receptors (NLRs), RIG-I-like receptor, C-type Lectin receptors (CLRs), and stimulator of interferon genes (STING). An “agonist” is a chemical that binds to a receptor and activates the receptor to produce a biological response. Agonists of the PPRs enhance immune responses (e.g., innate or adaptive immune response). Agonists of PPRs are known to those skilled in the art. For example, various TLR and NLR agonists are described in Kaur et al., Curr. Opin. Chem. Biol. 2022, 70, 102172; Kaczanowska et al, J Leukoc Biol. 2013 June; 93(6): 847-863; Higgins et al., Curr Infect Dis Rep. 2010 January; 12(1):4-12; and Maisonneuve et al., Proc Natl Acad Sci USA. 2014 Aug. 26; 111(34): 12294-12299, incorporated herein by reference. RIG-I-like receptor agonists are described in Bourquin, et al., Pharmacological Research, 2020, 154, 104192; Ranjith-Kumar et al., J Biol Chem. 2009 Jan. 9; 284(2): 1155-1165; and Goulet et al., PLOS Pathogens 9(8): 10, incorporated herein by reference. CLR agonists are described in Lamb et al., Biochemistry. 2002 Dec. 3; 41(48):14340-7; and Yan et al., Front Immunol. 2015; 6: 408, incorporated herein by reference. STING agonists are described in Amouzegar, et al., Cancers 2021, 13, 2695; Fu et al., Sci Transl Med. 2015 Apr. 15; 7(283): 283ra52; and Foote et al., Cancer Immunology Research, DOI: 10.1158/2326-6066.CIR-16-0284, incorporated herein by reference. The PPR agonists described herein are also commercially available, e.g., from InvivoGen (California, USA). In some embodiments, the second adjuvant is alum.

In some embodiments, the vaccine compositions described herein are formulated for administration to a subject. In some embodiments, the vaccine composition is formulated or administered in combination with one or more pharmaceutically acceptable excipients. In some embodiments, vaccine compositions comprise at least one additional active substance such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

Formulations of the vaccine compositions described herein may be prepared by any method known or hereafter developed in the art of pharmacology. In general, such preparatory methods include the step of bringing the antigen and/or the adjuvant (e.g., Compound (1)) into association with an excipient (e.g., pharmaceutically acceptable excipient) and/or one or more other accessory ingredients, and then, if necessary and/or desirable, dividing, shaping and/or packaging the product into a desired single- or multi-dose unit.

Relative amounts of the antigen, the adjuvant, the pharmaceutically acceptable excipient, and/or any additional ingredients in a vaccine composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

The antigen, Compound (1), and/or optionally the second adjuvant may be formulated using any of the methods described herein or known in the art separately or together.

### Methods of Treatment and Uses

Compound (1) is capable of enhancing an immune response (e.g., innate and/or adaptive immune response). The present disclosure thus also provides methods of enhancing an immune response (e.g., innate and/or adaptive immune response) in a subject, biological sample, tissue, or cell. The present disclosure further provides methods for Compound (1) as adjuvants in a vaccine for treatment of a wide range of diseases, such as proliferative diseases, inflammatory diseases, autoimmune diseases, infectious diseases, addiction, and chronic diseases in a subject in need thereof, or as stand alone or immune response modifying agents.

In another aspect, the present disclosure provides methods of enhancing an immune response (e.g., innate and/or adaptive immune response), the methods comprising administering to the subject an effective amount of Compound (1) or pharmaceutical composition described herein.

In another aspect, the present disclosure provides methods of enhancing an immune response (e.g., innate and/or adaptive immune response) in a biological sample, tissue, or cell, the methods comprising contacting the biological sample, tissue, or cell with an effective amount of Compound (1) or pharmaceutical composition described herein.

In certain embodiments, the immune response (e.g., innate and/or adaptive immune response) is enhanced by a compound, pharmaceutical composition, kit, use, or method described herein by at least 1%, at least 3%, at least 10%, at least 20%, at least 30%, at least 40%, at least 50%, at least 60%, at least 70%, at least 80%, or at least 90%. In certain embodiments, the immune response (e.g., innate and/or adaptive immune response) in a subject, biological sample, tissue, or cell is enhanced by a compound, pharmaceutical composition, kit, use, or method described herein by not more than 1%, not more than 3%, not more than 10%, not more than 20%, not more than 30%, not more than 40%, not more than 50%, not more than 60%, not more than 70%, not more than 80%, or not more than 90%.

In some embodiments, the methods comprise administering to the subject an effective amount of Compound (1) (e.g., for enhancing an innate immune response, including induction of heterologous or trained immunity or innate memory).

Another aspect of the present disclosure relates to methods of using Compound (1) as an adjuvant in a vaccine for treating a disease in a subject in need thereof, the methods comprising administering to the subject a therapeutically effective amount of the compound or pharmaceutical composition described herein. In some embodiments, the methods comprising administering to the subject an effective amount of Compound (1) and an effective amount of an antigen (e.g., for enhancing an antigen-specific immune response). In some embodiments, Compound (1) is administered separately from the antigen. In some embodiments, Compound (1) is administered prior to administering the antigen. In some embodiments, Compound (1) is administered after administering the antigen. In some embodiments, Compound (1) and the antigen are administered simultaneously. In some embodiments, Compound (1) and the antigen are administered as an admixture.

The antigen and/or Compound (1) (e.g., the antigen alone, Compound (1) alone, or the antigen and Compound (1) together) described herein elicits an immune response in the subject. In some embodiments, the antigen and/or Compound (1) activates cytokine and/or chemokine (e.g., CXCL-8) production. In some embodiments, the immune response is an innate immune response. In some embodiments, the immune response is an adaptive immune response specific to the antigen in the composition or vaccine. In some embodiments, the antigen and/or Compound (1) activates B cell immunity. In some embodiments, the antigen and/or Compound (1) elicits antibody production. In some embodiments, the composition and/or the vaccine activates cytotoxic T cells specific to the antigen.

In some embodiments, Compound (1), whether administered alone or in an admixture with an antigen, enhances the innate immune response, compared to without Compound (1) or when the antigen is administered alone. In some embodiments, Compound (1) activates peripheral blood mononuclear cells (PBMCs). In some embodiments, the number of PBMCs that are activated is increased by at least 10% in the presence of Compound (1), compared to without Compound (1) or when the antigen is administered alone. For example, the number of PBMCs that are activated may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, or at least 1000-fold or more, in the presence of Compound (1), compared to without Compound (1) or when the antigen is administered alone. In some embodiments, the number of PBMCs that are activated is increased by 20%, 30%, 40%, 50%, 60%, 70%, 80%, 90%, 100%, 2-fold, 5-fold, 10-fold, 100-fold, 1000-fold or more, in the presence of Compound (1), compared to without Compound (1) or when the antigen is administered alone. In some embodiments, Compound (1) enhances response in PBMCs. In some embodiments, Compound (1) increases response in PBMCs by 20%, 30%, 40%, 50%, 60%, 70%, 80%, 90%, 100%, 2-fold, 5-fold, 10-fold, 100-fold, 1000-fold or more, in the presence of Compound (1), compared to without Compound (1) or when the antigen is administered alone.

In some embodiments, Compound (1) activates a pattern recognition receptor (PRR). In some embodiments, Compound (1) activates one or more pattern recognition receptors (PRRs). In some embodiments, the PRR is selected from the group consisting of toll-like receptors (e.g., TLR2), NOD1/2, RIG-1/MDA-5, C-type lectins, and STING. In some embodiments, the Toll-like receptor is TLR1, TLR2, TLR3, TLR4, TLR5, TLR6, TLR9, and/or TLR10. In some embodiments, the Toll-like receptor is TLR7 and/or TLR8. In some embodiments, the Toll-like receptor is TLR7. In some embodiments, the number of PRRs that are activated is increased by at least 20% in the presence of Compound (1), compared to without Compound (1) or when the antigen is administered alone. For example, the number of PRRs that are activated may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, or at least 1000-fold or more, in the presence of Compound (1), compared to without Compound (1) or when the antigen is administered alone. In some embodiments, the number of PRRs that are activated is increased by 20%, 30%, 40%, 50%, 60%, 70%, 80%, 90%, 100%, 2-fold, 5-fold, 10-fold, 100-fold, 1000-fold or more, in the presence of Compound (1), compared to without Compound (1) or when the antigen is administered alone.

In some embodiments, Compound (1) induces the production of immunomodulatory cytokines and/or chemokines in the subject. In some embodiments, the immunomodulatory cytokines and/or chemokines are proinflammatory cytokines and/or chemokines. In some embodiments, the cytokines and chemokines include TNF, IL-12, IL-6, IL-1-β, or CXCL-8. In some embodiments, the level of immunomodulatory cytokines and/or chemokines (e.g., CXCL-8) is increased by at least 20% in the presence of Compound (1), compared to without Compound (1) or when the antigen is administered alone. For example, the level of immunomodulatory cytokines and/or chemokines (e.g., CXCL8) may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, or at least 1000-fold or more, in the presence of Compound (1), compared to Compound (1) or when the antigen is administered alone. In some embodiments, the level of immunomodulatory cytokines and/or chemokines (e.g., CXCL8) is increased by 20%, 30%, 40%, 50%, 60%, 70%, 80%, 90%, 100%, 2-fold, 5-fold, 10-fold, 100-fold, 1000-fold or more, in the presence of Compound (1), compared to without Compound (1) or when the antigen is administered alone.

In some embodiments, Compound (1) enhances innate immune memory (also referred to as trained immunity). “Innate immune memory” confers heterologous immunity that provides broad protection against a range of pathogens. In some embodiments, the innate immune memory is increased by at least 20% in the presence of Compound (1), compared to without Compound (1) or when the antigen is administered alone. For example, the innate immune memory may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, or at least 1000-fold or more, in the presence of Compound (1), compared to without Compound (1) or when the antigen is administered alone. In some embodiments, the innate immune memory is increased by 20%, 30%, 40%, 50%, 60%, 70%, 80%, 90%, 100%, 2-fold, 5-fold, 10-fold, 100-fold, 1000-fold or more, in the presence of Compound (1), compared to without Compound (1) or when the antigen is administered alone.

In some embodiments, Compound (1), when administered as an admixture with an antigen (e.g., the vaccine composition described herein), enhances the antigen-specific immune response against the antigen or against the invading agent where the antigen is derived from e.g., a microbial pathogen or cancer, compared to without Compound (1) (i.e., when the antigen is administered alone). In some embodiments, Compound (1) enhances the production of antigen-specific antibody titer (e.g., by at least 20%) in the subject, compared to without Compound (1) (i.e., when the antigen is administered alone). For example, Compound (1) may enhance the production of antigen-specific antibody titer by at least 20%, at least 30%, at least 40%, at least 50%, at least 60%, at least 70%, at least 80%, at least 90%, at least 100%, at least 2-fold, at least 5-fold, at least 10-fold, at least 100-fold, or at least 1000-fold or more, in the subject, compared to without Compound (1) (i.e., when the antigen is administered alone). In some embodiments, Compound (1) enhances the production of antigen-specific antibody titer by 20%, 30%, 40%, 50%, 60%, 70%, 80%, 90%, 100%, 2-fold, 5-fold, 10-fold, 100-fold, or 1000-fold or more, in the presence of Compound (1), compared to without Compound (1) (i.e., when the antigen is administered alone). One skilled in the art is familiar with how to evaluate the level of an antibody titer, e.g., by ELISA.

In some embodiments, Compound (1) enhances the activation of cytotoxic T-cells (e.g., by at least 20%) in the subject, compared to without Compound (1) (i.e., when the antigen is administered alone). For example, Compound (1) may enhance activation of cytotoxic T-cells by at least 20%, at least 30%, at least 40%, at least 50%, at least 60%, at least 70%, at least 80%, at least 90%, at least 100%, at least 2-fold, at least 5-fold, at least 10-fold, at least 100-fold, or at least 1000-fold or more, in the subject, compared to without Compound (1) (i.e., when the antigen is administered alone). In some embodiments, Compound (1) enhances the activation of cytotoxic T-cells by 20%, 30%, 40%, 50%, 60%, 70%, 80%, 90%, 100%, 2-fold, 5-fold, 10-fold, 100-fold, or 1000-fold or more, in the presence of Compound (1), compared to without Compound (1) (i.e., when the antigen is administered alone).

In some embodiments, Compound (1) polarizes the innate and adaptive immune response by shaping the pattern of cytokine and/or chemokine responses toward T helper 1 (Th1) immunity, which is important for host defense against intracellular pathogens. In some embodiments, Compound (1) polarizes the innate immune response toward T helper 2 (Th2) immunity, which is important for humoral immunity including antibody production. In some embodiments, humoral responses polarize the innate immune response toward T helper 17 (Th17) immunity, which is important for mucosal immunity. In some embodiments, Compound (1) polarizes the innate immune response toward T follicular helper (Tfh) cell immunity.

The innate immune system plays a crucial role in the control of initiation of the adaptive immune response and in the induction of appropriate cell effector responses. (Fearon et al. (1996) Science 272: 50-3; Medzhitov et al. (1997) Cell 91: 295-8, incorporated herein by reference). As such, in some embodiments, Compound (1) enhances the innate immune response in a subject (e.g., when administered alone or in an admixture with an antigen), which in turn enhances the immunogenicity of a given antigen. This is particularly useful in subjects that have an underdeveloped (e.g., in a neonatal infant), weak (e.g., in older adults), compromised immune system (e.g., in a patient with primary immunodeficiency or acquired immunodeficiency secondary to HIV patient infection or a patient with cancer receiving chemotherapy and/or radiation therapy) or in those with a distinct immune system (e.g., in a subject with history of substance use disorder etc.).

In some embodiments, Compound (1) prolongs the durability of protection of a vaccine (e.g., by at least 20%) in the subject, compared to without Compound (1) (i.e., when the antigen is administered alone). For example, Compound (1) may prolong the effect of a vaccine by at least 20%, at least 30%, at least 40%, at least 50%, at least 60%, at least 70%, at least 80%, at least 90%, at least 100%, at least 2-fold, at least 5-fold, at least 10-fold, at least 100-fold, or at least 1000-fold or more, in the subject, compared to without Compound (1) (i.e., when the antigen is administered alone). In some embodiments, Compound (1) prolongs the effect of a vaccine by 20%, 30%, 40%, 50%, 60%, 70%, 80%, 90%, 100%, 2-fold, 5-fold, 10-fold, 100-fold, or 1000-fold or more, in the presence of Compound (1), compared to without Compound (1) (i.e., when the antigen is administered alone).

In some embodiments, Compound (1) increases rate of (accelerates) an immune response, compared to without Compound (1) (i.e., when the antigen is administered alone). For example, Compound (1) may increase the rate of an immune response by at least 20%, at least 30%, at least 40%, at least 50%, at least 60%, at least 70%, at least 80%, at least 90%, at least 100%, at least 2-fold, at least 5-fold, at least 10-fold, at least 100-fold, or at least 1000-fold or more, in the subject, compared to without Compound (1) (i.e., when the antigen is administered alone). In some embodiments, Compound (1) increases the rate of an immune response by 20%, 30%, 40%, 50%, 60%, 70%, 80%, 90%, 100%, 2-fold, 5-fold, 10-fold, 100-fold, 1000-fold or more, in the presence of Compound (1), compared to without Compound (1) (i.e., when the antigen is administered alone). “Increase the rate of immune response” means that in the presence of the compound it takes less time for the immune system of a subject to react to an invading agent (e.g., a microbial pathogen).

In some embodiments, the presence of Compound (1) enables antigen dose sparing—i.e., enables a given antigen to induce the same immunogenicity at a lower antigen dose compared to without Compound (1) (i.e., when the antigen is administered alone). In some embodiments, the amount of antigen needed to produce the same level of immune response is reduced by at least 20% in the presence of Compound (1), compared to without Compound (1) (i.e., when the antigen is administered alone). For example, the amount of antigen needed to produce the same level of immune response may be reduced by at least 20%, at least 30%, at least 40%, at least 50%, at least 60%, at least 70%, at least 80%, at least 90%, at least 95%, at least 99% or more, in the presence of Compound (1), compared to without Compound (1) (i.e., when the antigen is administered alone). In some embodiments, the amount of antigen needed to produce the same level of immune response is reduced by at 20%, 30%, 40%, 50%, 60%, 70%, 80%, 90%, 95%, 99% or more, in the presence of Compound (1), compared to without Compound (1) (i.e., when the antigen is administered alone).

The prophylactic or therapeutic use of Compound (1), or a composition or vaccine composition described herein is also within the scope of the present disclosure. In some embodiments, the composition or vaccine composition described herein are used in methods of vaccinating a subject by prophylactically administering to the subject an effective amount of the composition or vaccine composition described herein. “Vaccinating a subject” refers to a process of administering an immunogen, typically an antigen formulated into a vaccine, to the subject in an amount effective to increase or activate an immune response against the antigen and, thus, against a pathogen displaying the antigen. In some embodiments, the terms do not require the creation of complete immunity against the pathogen. In some embodiments, the terms encompass a clinically favorable enhancement of an immune response toward the antigen or pathogen. Methods for immunization, including formulation of a vaccine composition and selection of doses, routes of administration and the schedule of administration (e.g., primary dose and one or more booster doses), are well known in the art. In some embodiments, vaccinating a subject reduces the risk of developing a disease (e.g., an infectious disease or cancer) in a subject.

In certain embodiments, the present disclosure provides methods of treating a disease. In certain embodiments, the disease is a proliferative disease, an inflammatory disease, an autoimmune disease, an infectious disease, an allergy, a fibrotic disease, a cardiovascular disease, a graft rejection, or graft-versus-host disease. In certain embodiments, the disease is a proliferative disease. In certain embodiments, the disease is cancer. Vaccine compositions comprising cancer-specific antigens and Compound (1) may be used in cancer immunotherapy by eliciting cancer-specific immune response against the cancer. In certain embodiments, the disease is hyperplasia (e.g., germinal center (GC) hyperplasia). In certain embodiments, the cancer is skin cancer, brain cancer, breast cancer, or prostate cancer. In certain embodiments, the cancer is melanoma. In certain embodiments, the disease is a benign neoplasm. In certain embodiments, the disease is or is associated with pathological angiogenesis.

In some embodiments, additional anti-cancer agents may be administered in combination with the compound, composition or vaccine composition described herein. In some embodiments, the anti-cancer agent is selected from the group consisting of small molecules, oligonucleotides, polypeptides, and combinations thereof. In some embodiments, the anti-cancer agent is a chemotherapeutic agent. In some embodiments, the chemotherapeutic agent is selected from the group consisting of: Actinomycin, All-trans retinoic acid, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vinblastine, Vincristine, Vindesine, and Vinorelbine. In some embodiments, the chemotherapeutic agent is Doxorubicin.

In some embodiments, the anti-cancer agent is an immune checkpoint inhibitor. An “immune checkpoint” is a protein in the immune system that either enhances an immune response signal (co-stimulatory molecules) or reduces an immune response signal. Many cancers protect themselves from the immune system by exploiting the inhibitory immune checkpoint proteins to inhibit the T cell signal. Exemplary inhibitory checkpoint proteins include, without limitation, Cytotoxic T-Lymphocyte-Associated protein 4 (CTLA-4), Programmed Death 1 receptor (PD-1), T-cell Immunoglobulin domain and Mucin domain 3 (TIM3), Lymphocyte Activation Gene-3 (LAG3), V-set domain-containing T-cell activation inhibitor 1 (VTVN1 or B7-H4), Cluster of Differentiation 276 (CD276 or B7-H3), B and T Lymphocyte Attenuator (BTLA), Galectin-9 (GAL9), Checkpoint kinase 1 (Chkl), Adenosine A2A receptor (A2aR), Indoleamine 2,3-dioxygenase (IDO), Killer-cell Immunoglobulin-like Receptor (KIR), Lymphocyte Activation Gene-3 (LAG3), and V-domain Ig suppressor of T cell activation (VISTA).

Some of these immune checkpoint proteins need their cognate binding partners, or ligands, for their immune inhibitory activity. For example, A2AR is the receptor of adenosine A2A and binding of A2A to A2AR activates a negative immune feedback loop. As another example, PD-1 associates with its two ligands, PD-L1 and PD-L2, to down regulate the immune system by preventing the activation of T-cells. PD-1 promotes the programmed cell death of antigen specific T-cells in lymph nodes and simultaneously reduces programmed cell death of suppressor T cells, thus achieving its immune inhibitory function. As yet another example, CTLA4 is present on the surface of T cells, and when bound to its binding partner CD80 or CD86 on the surface of antigen-present cells (APCs), it transmits an inhibitory signal to T cells, thereby reducing the immune response.

An “immune checkpoint inhibitor” is a molecule that prevents or weakens the activity of an immune checkpoint protein, for example, an immune checkpoint inhibitor may inhibit the binding of the immune checkpoint protein to its cognate binding partner, e.g., PD-1, CTLA-4, or A2aR. In some embodiments, the immune checkpoint inhibitor is a small molecule. In some embodiments, the immune checkpoint inhibitor is a nucleic acid aptamer (e.g., a siRNA targeting any one of the immune checkpoint proteins). In some embodiments, the immune checkpoint inhibitor is a recombinant protein. In some embodiments, the immune checkpoint inhibitor is an antibody. In some embodiments, the antibody comprises an anti-CTLA-4, anti-PD-1, anti-PD-L1, anti-TIM3, anti-LAG3, anti-B7-H3, anti-B7-H4, anti-BTLA, anti-GAL9, anti-Chk, anti-A2aR, anti-IDO, anti-KIR, anti-LAG3, anti-VISTA antibody, or a combination of any two or more of the foregoing antibodies. In some embodiments, the immune checkpoint inhibitor is a monoclonal antibody. In some embodiments, the immune checkpoint inhibitor comprises anti-PDi, anti-PD-L1, anti-CTLA-4, or a combination of any two or more of the foregoing antibodies. For example, the anti-PD-1 antibody is pembrolizumab (Keytruda®) or nivolumab (Opdivo®) and the anti-CTLA-4 antibody is ipilimumab (Yervoy®). Thus, in some embodiments, the immune checkpoint inhibitor comprises pembrolizumab, nivolumab, ipilimumab, or any combination of two or more of the foregoing antibodies. The examples described herein are not meant to be limiting and that any immune checkpoint inhibitors known in the art and any combinations thereof may be used in accordance with the present disclosure.

Additional exemplary agents that may be used in combination with the compound and compositions described herein include, but are not limited to, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, and a combination thereof. In some embodiments, the additional agent is an anti-proliferative agent (e.g., anti-cancer agent). In some embodiments, the additional pharmaceutical agent is an anti-leukemia agent. In certain embodiments, the additional pharmaceutical agent is an anti-lymphoma agent. In certain embodiments, the additional pharmaceutical agent is ABITREXATE (methotrexate), ABRAXANE (paclitaxel albumin-stabilized nanoparticle formulation), ABVD, ABVE, ABVE-PC, ADCETRIS (brentuximab vedotin), ADRIAMYCIN RDF (doxorubicin hydrochloride), AMBOCHLORIN (chlorambucil), ARRANON (nelarabine), AC, AC-T, ADE, ADRIAMYCIN PFS (doxorubicin hydrochloride), ADRUCIL (fluorouracil), AFINITOR (everolimus), AFINITOR DISPERZ (everolimus), ALDARA (imiquimod), ALIMTA (pemetrexed disodium), AREDIA (pamidronate disodium), ARIMIDEX (anastrozole), AROMASIN (exemestane), ARZERRA (ofatumumab), AVASTIN (bevacizumab), BEACOPP, BECENUM (carmustine), BELEODAQ (belinostat), BEP, BICNU (carmustine), BLENOXANE (bleomycin), BOSULIF (bosutinib), BUSULFEX (busulfan), CAF, CAMPATH (alemtuzumab), CLOFAREX (clofarabine), CLOLAR (clofarabine), CVP, CAMPTOSAR (irinotecan hydrochloride), CAPOX, CAPRELSA (vandetanib), CARBOPLATIN-TAXOL, CARMUBRIS (carmustine), CASODEX (bicalutamide), CEENU (lomustine), CERUBIDINE (daunorubicin hydrochloride), CERVARIX (recombinant HPV bivalent vaccine), CHOP, CLAFEN (cyclophosphamide), CMF, COMETRIQ (cabozantinib-s-malate), COPP, COPP-ABV, CVP, COSMEGEN (dactinomycin), CYFOS (ifosfamide), CYRAMZA (ramucirumab), CYTOSAR-U (cytarabine), CYTOXAN (cyclophosphamide), DACOGEN (decitabine), DEGARELIX, DEPOCYT (liposomal cytarabine), DTIC-DOME (dacarbazine), DOXIL (doxorubicin hydrochloride liposome), DOXORUBICIN HYDROCHLORIDE, DOX-SL (doxorubicin hydrochloride liposome), DTIC-DOME (dacarbazine), EFUDEX (fluorouracil), ELLENCE (epirubicin hydrochloride), ELOXATIN (oxaliplatin), EPOCH, ERBITUX (cetuximab), ERIVEDGE (vismodegib), ERWINAZE (Asparaginase Erwinia Chrysanthemi), ETOPOPHOS (etoposide phosphate), EVACET (doxorubicin hydrochloride liposome), EXXAR (tositumomab and iodine I 131 tositumomab), FARESTON (toremifene), FASLODEX (fulvestrant), FEC, FEMARA (letrozole), FLUDARA (fludarabine phosphate), FLUOROPLEX (fluorouracil), FOLEX (methotrexate), FOLEX PFS (methotrexate), FOLOTYN (pralatrexate), FOLFIRI, FOLFIRI-BEVACIZUMAB, FOLFIRI-CETUXIMAB, FOLFIRINOX, FOLFOX, FU-LV, GARDASIL (recombinant human papillomavirus (HPV) quadrivalent vaccine), GAZYVA (obinutuzumab), GEMCITABINE-CISPLATIN, GEMCITABINE-OXALIPLATIN, GEMZAR (gemcitabine hydrochloride), GILOTRIF (afatinib dimaleate), GLEEVEC (imatinib mesylate), GLIADEL (carmustine implant), GLIADEL WAFER (carmustine implant), HERCEPTIN (trastuzumab), HYCAMTIN (topotecan hydrochloride), Hyper-CVAD, ICE, ISTODAX (romidepsin), ICLUSIG (ponatinib hydrochloride), IDAMYCIN (idarubicin), IMBRUVICA (ibrutinib), IFEX (ifosfamide), IFOSFAMIDUM (ifosfamide), INLYTA (axitinib), INTRON A (recombinant interferon alfa-2b), IRESSA (gefitinib), IXEMPRA (ixabepilone), JAKAFI (ruxolitinib phosphate), JEVTANA (cabazitaxel), KADCYLA (ado-trastuzumab emtansine), KEYTRUDA (pembrolizumab), KYPROLIS (carfilzomib), Lomustine, LEUKERAN (chlorambucil), LEUSTATIN (cladribine), LINFOLIZIN (chlorambucil), LIPODOX (doxorubicin hydrochloride liposome), LUPRON (leuprolide acetate), LUPRON DEPOT (leuprolide acetate), LUPRON DEPOT-3 MONTH (leuprolide acetate), LUPRON DEPOT-4 MONTH (leuprolide acetate), LUPRON DEPOT-PED (leuprolide acetate), MATULANE (procarbazine hydrochloride), MOPP, MOZOBIL (plerixafor), MARQIBO (vincristine sulfate liposome), MYLERAN (busulfan), MEGACE (megestrol acetate), MEKINIST (trametinib), METHAZOLASTONE (temozolomide), METHOTREXATE LPF (methotrexate), MEXATE (methotrexate), MEXATE-AQ (methotrexate), MITOXANTRONE HYDROCHLORIDE, MITOZYTREX (mitomycin c), MOZOBIL (plerixafor), MUSTARGEN (mechlorethamine hydrochloride), MUTAMYCIN (mitomycin c), MYLOSAR (azacitidine), NAVELBINE (vinorelbine tartrate), NEOSAR (cyclophosphamide), NEXAVAR (sorafenib tosylate), NOLVADEX (tamoxifen citrate), NOVALDEX (tamoxifen citrate), OEPA, ONTAK (denileukin diftitox), OPPA, OFF, ONCASPAR (Pegaspargase), PAD, PARAPLAT (carboplatin), PARAPLATIN (carboplatin), PEG-INTRON (peginterferon alfa-2b), PEMETREXED DISODIUM, PERJETA (pertuzumab), PLATINOL (cisplatin), PLATINOL-AQ (cisplatin), POMALYST (pomalidomide), prednisone, PROLEUKIN (aldesleukin), PROLIA (denosumab), PROVENGE (sipuleucel-t), PURINETHOL (mercaptopurine), PURIXAN (mercaptopurine), REVLIMID (lenalidomide), R-CHOP, RUBIDOMYCIN (daunorubicin hydrochloride), RITUXAN (rituximab), SPRYCEL (dasatinib), STANFORD V, SYNRIBO (omacetaxine mepesuccinate), STIVARGA (regorafenib), SUTENT (sunitinib malate), SYLATRON (peginterferon alfa-2b), SYLVANT (siltuximab), SYNOVIR (thalidomide), TAC, TAFINLAR (dabrafenib), TARABINE PFS (cytarabine), TARCEVA (erlotinib hydrochloride), TASIGNA (nilotinib), TAXOL (paclitaxel), TAXOTERE (docetaxel), TEMODAR (temozolomide), THALOMID (thalidomide), TOPOSAR (etoposide), TORISEL (temsirolimus), TPF, TREANDA (bendamustine hydrochloride), TRISENOX (arsenic trioxide), TYKERB (lapatinib ditosylate), VAMP, VECTIBIX (panitumumab), VEIP, VELBAN (vinblastine sulfate), VELCADE (bortezomib), VELSAR (vinblastine sulfate), VEPESID (etoposide), VIADUR (leuprolide acetate), VIDAZA (azacitidine), VINCASAR PFS (vincristine sulfate), VOTRIENT (pazopanib hydrochloride), WELLCOVORIN (leucovorin calcium), XALKORI (crizotinib), XELODA (capecitabine), XELOX, XGEVA (denosumab), XOFIGO (radium 223 dichloride), XTANDI (enzalutamide), YERVOY (ipilimumab), ZALTRAP (ziv-aflibercept), ZELBORAF (vemurafenib), ZEVALIN (ibritumomab tiuxetan), ZOLINZA (vorinostat), ZOLADEX (goserelin acetate), ZOMETA (zoledronic acid), ZYDELIG (idelalisib), ZYKADIA (ceritinib), ZYTIGA (abiraterone acetate), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (Velcade)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine, or a combination thereof. In certain embodiments, the additional pharmaceutical agent is a binder or inhibitor of an HMT (e.g., EZH1, EZH2, DOT1). In certain embodiments, the additional pharmaceutical agent is a protein kinase inhibitor (e.g., tyrosine protein kinase inhibitor). In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation.

In certain embodiments, the disease is an inflammatory disease. In certain embodiments, the disease is an autoimmune disease. In certain embodiments, the disease is an allergic or atopic disease.

In certain embodiments, the disease is an infectious disease. In certain embodiments, the disease is a viral infectious disease, sepsis, a pediatric infectious disease, a bacterial infectious disease, a mycobacterial infectious disease, a parasitic infectious disease, or a fungal infectious disease. In certain embodiments, the disease is an infection by a microbial pathogen (e.g., from a mycobacterium, bacterium, fungus, a virus, parasite, or prion). In certain embodiments, a disease described herein is a microbial infectious disease. In certain embodiments, the infectious disease is a viral infectious disease. In certain embodiments, the viral infectious disease being treated or prevented is an infection caused by influenza. In certain embodiments, the viral infectious disease being treated or prevented is an infection caused by a coronavirus. In certain embodiments, the viral infectious disease being treated or prevented is an infection caused by SARS-CoV-2. In certain embodiments, the infectious disease is sepsis. In certain embodiments, the infectious disease is a pediatric infectious disease. In certain embodiments, the infectious disease is a disease of newborns, infants and/or school age children.

In some embodiments, the infectious disease is caused by Plasmodium malariae (malaria), Bacillus anthracis (anthrax), Bordetella pertussis (whooping cough), Corynebacterium diphtheriae (diphtheria), Clostridium tetani (tetanus), Haemophilus influenzae type b, pneumococcus (pneumococcal infections), Staphylococci spp., Group A or B streptococci, Mycobacterium tuberculosis, Neiserria meningitidis (meningococcal disease), Salmonella typhi (typhoid), Vibrio cholerae (Cholera), or Yersinia pestis (plague). In some embodiments, the infectious disease is caused by adenovirus, enterovirus such as polio virus, Ebola virus, herpes viruses (e.g., herpes simplex virus 1, herpes simplex virus 2, human herpesvirus 6, human herpesvirus 8), cytomegalovirus and varicella-zoster (chickenpox and shingles), measles, mumps, rubella, hepatitis-A, -B, or -C, human papilloma virus, Influenza virus, parainfluenza virus, a coronavirus (e.g., SARS-CoV-2), rabies, Japanese encephalitis, rotavirus, human immunodeficiency virus (HIV), respiratory syncytial virus (RSV), smallpox, monkeypox, yellow fever, or Zika Virus. In some embodiments, the infectious disease is caused by malaria, Leishmania, or a helminth. In some embodiments, the infectious disease is caused by Candida spp., Aspergillus spp., Cryptococcus spp., Mucoromycete, Blastomyces dermatitidis, Histoplasma capsulatum, or Sporothrix schenckii. In some embodiments, the infectious disease is caused by prion.

In certain embodiments, the infectious disease is a bacterial infectious disease. In certain embodiments, the bacterial infectious disease being treated or prevented is caused by an infection with a Gram-positive bacteria including, but not limited to, Staphylococcus spp., Streptococcus spp., Micrococcus spp., Peptococcus spp., Peptostreptococcus spp., Enterococcus spp., Bacillus spp., Clostridium spp., Lactobacillus spp., Listeria spp., Erysipelothrix spp., Propionibacterium spp., Eubacterium spp., Corynebacterium spp., Capnocytophaga spp., Bifidobacterium spp., and Gardnerella spp. In certain embodiments, the Gram-positive bacteria is a bacterium of the phylum Firmicutes. In certain embodiments, the Gram-positive bacteria is Streptococcus.

In certain embodiments, the bacterial infection being treated or prevented is an infection caused by a Gram-negative bacteria including, but not limited to, Escherichia, Citrobacter, Enterobacter, Klebsiella, Proteus, Serratia, Shigella, Salmonella, Morganella, Providencia, Edwardsiella, Erwinia, Hafnia, Yersinia, Acinetobacter, Vibrio, Aeromonas, Pseudomonas, Haemophilus, Pasteurella, Campylobacter, Helicobacter, Branhamella, Moraxella, Neisseria, Veillonella, Fusobacterium, Bacteroides, Actinobacillus, Aggregatibacter, Agrobacterium, Porphyromonas, Prevotella, Ruminobacter, Roseburia, Caulobacter, Francisella, Borrelia, Treponema, Brucella, Bordetella, and Rickettsia. In some embodiments, the bacterial infectious agent is Bacillus anthracis (causing anthrax), Bordetella pertussis (causing whooping cough), Corynebacterium diphtheriae (causing diphtheria), Clostridium tetani (causing tetanus), Haemophilus influenzae type b, pneumococcus (causing pneumococcal infections), Staphylococci spp. (including Group A or B streptococci), Mycobacterium tuberculosis, Neiserria meningitidis (causing meningococcal disease), Salmonella typhi (causing typhoid), Vibrio cholerae (causing Cholera), or Yersinia pestis (causing plague). In some embodiments, the bacterial infectious agent is anthrax, diphtheria, tetanus, Bordetella spp., Haemophilus influenzae type b, Neisseria spp., Vibrio spp., cholera, Yersinia spp., Staphylococci spp., Streptococci spp., or Salmonella spp. In some embodiments, the bacterial infectious disease is anthrax, diphtheria, tetanus, pertussis, Haemophilus influenzae type b, pneumococcal infection, meningococcal disease, cholera, plague, Staphylococcal disease, Group A or B streptococcal or pneumococcal infection, or typhoid.

In certain embodiments, the Gram-negative bacteria is Bordetella pertussis. In certain embodiments, the bacterial infectious disease is pertussis. In some embodiments, the antigen is a lipopolysaccharide endotoxin (LPS) from a Gram-negative bacterium. Non-limiting examples of Gram-negative bacterial species include: Neisseria species including Neisseria gonorrhoeae and Neisseria meningitidis, Branhamella species including Branhamella catarrhalis, Escherichia species including Escherichia coli, Enterobacter species, Proteus species including Proteus mirabilis, Pseudomonas species including Pseudomonas aeruginosa, Pseudomonas mallei, and Pseudomonas pseudomallei, Klebsiella species including Klebsiella pneumoniae, Salmonella species, Shigella species, Serratia species, Acinetobacter species; Haemophilus species including Haemophilus influenzae and Haemophilus ducreyi; Brucella species, Yersinia species including Yersinia pestis and Yersinia enterocolitica, Francisella species including Francisella tularensis, Pasteurella species including Pasteurella multocida, Vibrio cholerae, Flavobacterium species, meningosepticum, Campylobacter species including Campylobacter jejuni, Bacteroides species (oral, pharyngeal) including Bacteroides fragilis, Fusobacterium species including Fusobacterium nucleatum, Calyinniatobacterium granulomatis, Streptobacillus species including Streptobacillus moniliformis, Legionella species including Legionella pneumophila.

In certain embodiments, the bacteria are acid-fast bacilli, spirochetes, or actinomycetes, Examples of acid-fast bacilli include Mycobacterium species including Mycobacterium tuberculosis and Mycobacterium leprae. Examples of spirochetes include Treponema species including Treponema pallidum, Treponema pertenue, Borrelia species including Borrelia burgdorferi (Lyme disease), and Borrelia recurrentis, and Leptospira species. Examples of actinomycetes include: Actinomyces species including Actinomyces israelii, and Nocardia species including Nocardia asteroides.

In certain embodiments, the bacteria is Escherichia spp., Enterobacter spp. (e.g., Enterobacter cloacae), Salmonella spp. (e.g., Salmonella enteritidis, Salmonella typhi), Shigella spp., Pseudomonas spp. (e.g., Pseudomonas aeruginosa, Pseudomonas pachastrellae, Pseudomonas stutzeri), Moraxella spp. (e.g., Moraxella catarrhalis), Neisseria spp. (e.g., Neisseria gonorrhoeae, Neisseria meningitidis), Helicobacter spp., (e.g., Helicobacter pylori) Stenotrophomonas spp., Vibrio spp. (e.g., Vibrio cholerae), Legionella spp. (Legionella pneumophila), Hemophilus spp. (e.g., Hemophilus influenzae), Klebsiella spp. (e.g., Klebsiella pneumoniae), Proteus spp. (e.g., Proteus mirabilis), Serratia spp. (Serratia marcescens), Streptococcus spp., Staphylococcus spp., Corynebacterium spp., Listeria spp., Bacillus spp. (e.g., Bacillus anthracis) Bordetella spp. (e.g., Bordetella pertussis); Borrelia spp. (e.g., Borrelia burgdorferi); Brucella spp. (e.g., Brucella abortus, Brucella canis, Brucella melitensis, Brucella suis); Campylobacter spp. (e.g., Campylobacter jejuni); Chlamydia spp. and Chlamydophila spp. (e.g., Chlamydia pneumoniae, Chlamydia trachomatis, Chlamydophila psittaci); Clostridium spp. (e.g., Clostridium botulinum, Clostridium difficile, Clostridium perfringens, Clostridium tetani); Corynebacterium spp. (e.g., Corynebacterium diphtheriae); Enterococcus spp. (e.g., Enterococcus faecalis, Enterococcus faecium); Escherichia spp. (e.g., Escherichia coli, Enterotoxic E. coli, enteropathogenic E. coli; E. coli O157:H7); Francisella spp. (e.g., Francisella tularensis); Haemophilus spp. (e.g., Haemophilus influenzae); Helicobacter spp. (e.g., Helicobacter pylori); Legionella spp. (e.g., Legionella pneumophila); Leptospira spp. (e.g., Leptospira interrogans); Listeria spp. (e.g., Listeria monocytogenes); Mycobacterium spp. (e.g., Mycobacterium leprae, Mycobacterium tuberculosis, Mycobacterium ulcerans); Mycoplasma spp. (e.g., Mycoplasma pneumoniae); Neisseria spp. (e.g., Neisseria gonorrhoeae, Neisseria meningitidis); Pseudomonas spp. (e.g., Pseudomonas aeruginosa); Rickettsia spp. (e.g., Rickettsia rickettsii); Salmonella spp. (e.g., Salmonella typhi, Salmonella typhimurium); Shigella spp. (e.g., Shigella sonnei); Staphylococcus spp. (e.g., Staphylococcus aureus, Staphylococcus epidermidis, Staphylococcus saprophyticus); Streptococcus spp. (e.g., Streptococcus agalactiae, Streptococcus pneumoniae, Streptococcus pyogenes); Treponema spp. (e.g., Treponema pallidum); Pseudodiomarina spp. (e.g., P. maritima); Marinobacter spp. (e.g., Marinobacter hydrocarbonoclasticus, Marinobacter vinifirmus) Alcanivorax spp. (e.g., Alcanivorax dieselolei); Acetinobacter spp. (e.g., A. venetianus); Halomonas spp. (e.g., H. shengliensis); Labrenzia spp.; Microbulifer spp. (e.g., M. schleiferi); Shewanella spp. (e.g., S. algae); Vibrio spp. (e.g., Vibrio cholerae, Vibrio alginolyticus, Vibrio hepatarius); and Yersinia spp. (e.g., Yersinia pestis).

In certain embodiments, the disease is a fibrotic disease, a cardiovascular disease, a graft rejection, or graft-versus-host disease. In certain embodiments, the disease is a mycobacterial infectious disease. In certain embodiments, the mycobacterial infectious disease is caused by a tuberculosis infection or a non-tuberculous mycobacterial infection.

In certain embodiments, the infectious disease is a viral infectious disease. In certain embodiments, the viral infectious disease is an infection caused by retroviruses, human immunodeficiency viruses including HIV-1, HDTV-III, LAVE, HTLV-III/LAV, HIV-III, HIV-LP, Cytomegaloviruses (CMV), Picornaviruses, polio viruses, hepatitis A virus, enteroviruses, human Coxsackie viruses, rhinoviruses, echoviruses, Calciviruses, Togaviruses, equine encephalitis viruses, rubella viruses, Flaviruses, dengue viruses, encephalitis viruses, yellow fever viruses, Coronaviruses (e.g., SARS-CoV-2), Rhabdoviruses, vesicular stomatitis viruses, rabies viruses, Filoviruses, ebola virus, Paramyxoviruses, parainfluenza viruses, mumps virus, measles virus, respiratory syncytial virus (RSV), Orthomyxoviruses, influenza viruses, Bungaviruses, Hantaan viruses, phleboviruses and Nairo viruses, Arena viruses, hemorrhagic fever viruses, reoviruses, orbiviruses, rotaviruses, Birnaviruses, Hepadnaviruses, Hepatitis B virus, parvoviruses, Papovaviridae, papilloma viruses, polyoma viruses, Adenoviruses, varicella zoster virus, Poxviruses, variola viruses, vaccinia viruses, Irido viruses, African swine fever virus, delta hepatitis virus, non-A, non-B hepatitis virus, Hepatitis C, Norwalk viruses, astroviruses, and unclassified viruses. In certain embodiments, the viral infectious disease is an infection caused by adenovirus, polio virus, Ebola, herpes viruses (e.g., herpes simplex virus 1, herpes simplex virus 2, human herpesvirus 6, human herpesvirus 8), cytomegalovirus and varicella-zoster, measles, mumps, rubella, hepatitis A, hepatitis B, hepatitis C, human papilloma virus, Influenza, parainfluenza virus, rabies, Japanese encephalitis, rotavirus, human immunodeficiency virus, respiratory syncytial virus, smallpox, monkeypox, yellow fever, or Zika Virus. In certain embodiments, the viral infectious disease is polio, chickenpox, or shingles. In certain embodiments, the viral infectious disease is an infection caused by human immunodeficiency virus (HIV) or respiratory syncytial virus (RSV). In certain embodiments, the viral infectious disease is an infection caused by influenza. In certain embodiments, the viral infectious disease is an infection caused by a coronavirus (e.g., SARS-Cov-2).

In certain embodiments, the infectious disease is a parasitic infectious disease. In certain embodiments, the parasitic infectious disease is an infection caused by Leishmania, another protozoan, or a helminth. In certain embodiments, the parasitic infectious disease is caused by Plasmodium species, such as Plasmodium species including Plasmodium falciparum, Plasmodium malariae, Plasmodium ovale, Plasmodium vivax and Toxoplasma gondii. In certain embodiments, the parasitic infectious disease is caused by blood-borne and/or tissues parasites include Plasmodium species, Babesia species including babesia microti and Babesia divergens, Leishmania species including Leishmania tropica, Leishmania species, Leishmania braziliensis, Leishmania donovani, Trypanosoma species including Trypanosoma gambiense. Trypanosoma rhodesiense (African sleeping sickness), and Trypanosoma cruzi (Chagas' disease). In certain embodiments, the parasitic infectious disease is malaria or leishmaniasis.

In certain embodiments, the infectious disease is a fungal infectious disease. In certain embodiments, the fungal infectious disease is caused by Cryptococcus species including Cryptococcus neoformans, Histoplasma species including Histoplasma capsulatum, Coccidioides species including Coccidiodes immitis, Paracoccidioides species including Paracoccidioides brasiliensis, Blastomyces species including Blastomyces dermatitidis, Chlamydia species including Chlamydia trachomatis, Candida species including Candida albicans and Candida auris, Sporothrix species including Sporothrix schenckii, Aspergillus species, or fungi of mucormycosis. In some embodiments, the fungus is Candida spp., Aspergillus spp., Cryptococcus spp., Mucoromycete, Blastomyces dermatitidis (causing blastomycosis), or endemic mycosis causing fungus such as Histoplasma capsulatum (causing histoplasmosis), or Sporothrix schenckii (causing sporotrichosis).

In certain embodiments, the vaccine provides heterologous protection against a range of pathogens. Other medically relevant microorganisms have been described extensively in the literature, e.g., see C. G. A Thomas, Medical Microbiology, Bailliere Tindall, Great Britain 1983, incorporated herein by reference.

In certain embodiments, the disease is a chronic disease. In certain embodiments, the chronic disease is arthritis, cardiovascular disease such as heart disease, stroke, cancer (e.g., breast cancer or colon cancer), chronic respiratory diseases, diabetes, epilepsy, seizures, obesity, or an oral health problem.

In certain embodiments, the disease is addiction. In certain embodiments, the disease is addiction to a psychoactive substance (e.g., opioids).

In some embodiments, the compound, composition or vaccine composition may be administered in combination with another therapeutic agent for the infectious diseases. Such other therapeutic agents may be, without limitation: antibiotics, anti-viral agents, anti-fungal agents, or anti-parasitic agents. One skilled in the art is familiar with how to select or administer the additional therapeutic agent based on the disease to be treated.

In some embodiments, the disease is an allergy (e.g., allergic rhinitis) or asthma. It has been demonstrated that Th1/Th2 imbalance results in the clinical manifestation of allergy or asthma (e.g., as described in Ngoc et al., Curr Opin Allergy Clin Immunol. 2005 April; 5(2):161-6, incorporated herein by reference). In certain embodiments, Compound (1) restores Th1/Th2 balance and possesses ability to treat allergy or asthma.

In still another aspect, the present disclosure provides methods of preventing a disease to be treated with a compound or pharmaceutical composition described herein in a subject in need thereof, the methods comprising administering to the subject a prophylactically effective amount of a compound, composition, pharmaceutical composition, vaccine, or vaccine composition described herein.

In certain embodiments, the subject has any of the diseases described herein (e.g., a proliferative disease, an inflammatory disease, an autoimmune disease, an infectious disease, an allergy, a fibrotic disease, a cardiovascular disease, a graft rejection, graft-versus-host disease, chronic disease, or addiction). In some embodiments, the subject is at risk of developing any of the diseases described herein (e.g., a proliferative disease, an inflammatory disease, an autoimmune disease, an infectious disease, an allergy, a fibrotic disease, a cardiovascular disease, a graft rejection, graft-versus-host disease, chronic disease, or addiction). In some embodiments, administering the antigen and Compound (1) to a subject having a disease treats the disease (therapeutic use). In some embodiments, administering Compound (1) to a subject at risk of developing a disease reduces the likelihood (e.g., by 20%, 30%, 40%, 50%, 60%, 70%, 80%, 90%, 95%, 99% or more) of the subject developing the disease (prophylactic use).

In certain embodiments, the subject is a human. In some embodiments, the subject is a human neonate. In some embodiments, the subject is a human infant. In some embodiments, the human infant is a neonate that is less than or equal to 28 days of age. In some embodiments, the human infant is 0-28 days, 0-27 days, 0-26 days, 0-25 days, 0-24 days, 0-23 days, 0-22 days, 0-21 days, 0-20 days, 0-19 days, 0-18 days, 0-17 days, 0-16 days, 0-15 days, 0-14 days, 0-13 days, 0-12 days, 0-11 days, 0-10 days, 0-9 days, 0-8 days, 0-7 days, 0-6 days, 0-5 days, 0-4 days, 0-3 days, 0-2 days, 0-1 days, 0-12 hours, 0-6 hours, 0-2 hours, 0-1 hour, 1-28 days, 1-27 days, 1-26 days, 1-25 days, 1-24 days, 1-23 days, 1-22 days, 1-21 days, 1-20 days, 1-19 days, 1-18 days, 1-17 days, 1-16 days, 1-15 days, 1-14 days, 1-13 days, 1-12 days, 1-11 days, 1-10 days, 1-9 days, 1-8 days, 1-7 days, 1-6 days, 1-5 days, 1-4 days, 1-3 days, 1-2 days, 2-28 days, 2-27 days, 2-26 days, 2-25 days, 2-24 days, 2-23 days, 2-22 days, 2-21 days, 2-20 days, 2-19 days, 2-18 days, 2-17 days, 2-16 days, 2-15 days, 2-14 days, 2-13 days, 2-12 days, 2-11 days, 2-10 days, 2-9 days, 2-8 days, 2-7 days, 2-6 days, 2-5 days, 2-4 days, 2-3 days, 3-28 days, 3-27 days, 3-26 days, 3-25 days, 3-24 days, 3-23 days, 3-22 days, 3-21 days, 3-20 days, 3-19 days, 3-18 days, 3-17 days, 3-16 days, 3-15 days, 3-14 days, 3-13 days, 3-12 days, 3-11 days, 3-10 days, 3-9 days, 3-8 days, 3-7 days, 3-6 days, 3-5 days, 3-4 days, 4-28 days, 4-27 days, 4-26 days, 4-25 days, 4-24 days, 4-23 days, 4-22 days, 4-21 days, 4-20 days, 4-19 days, 4-18 days, 4-17 days, 4-16 days, 4-15 days, 4-14 days, 4-13 days, 4-12 days, 4-11 days, 4-10 days, 4-9 days, 4-8 days, 4-7 days, 4-6 days, 4-5 days, 5-28 days, 5-27 days, 5-26 days, 5-25 days, 5-24 days, 5-23 days, 5-22 days, 5-21 days, 5-20 days, 5-19 days, 5-18 days, 5-17 days, 5-16 days, 5-15 days, 5-14 days, 5-13 days, 5-12 days, 5-11 days, 5-10 days, 5-9 days, 5-8 days, 5-7 days, 5-6 days, 6-28 days, 6-27 days, 6-26 days, 6-25 days, 6-24 days, 6-23 days, 6-22 days, 6-21 days, 6-20 days, 6-19 days, 6-18 days, 6-17 days, 6-16 days, 6-15 days, 6-14 days, 6-13 days, 6-12 days, 6-11 days, 6-10 days, 6-9 days, 6-8 days, 6-7 days, 7-28 days, 7-27 days, 7-26 days, 7-25 days, 7-24 days, 7-23 days, 7-22 days, 7-21 days, 7-20 days, 7-19 days, 7-18 days, 7-17 days, 7-16 days, 7-15 days, 7-14 days, 7-13 days, 7-12 days, 7-11 days, 7-10 days, 7-9 days, 7-8 days, 9-28 days, 9-27 days, 9-26 days, 9-25 days, 9-24 days, 9-23 days, 9-22 days, 9-21 days, 9-20 days, 9-19 days, 9-18 days, 9-17 days, 9-16 days, 9-15 days, 9-14 days, 9-13 days, 9-12 days, 9-11 days, 9-10 days, 10-28 days, 10-27 days, 10-26 days, 10-25 days, 10-24 days, 10-23 days, 10-22 days, 10-21 days, 10-20 days, 10-19 days, 10-18 days, 10-17 days, 10-16 days, 10-15 days, 10-14 days, 10-13 days, 10-12 days, 10-11 days, 11-28 days, 11-27 days, 11-26 days, 11-25 days, 11-24 days, 11-23 days, 11-22 days, 11-21 days, 11-20 days, 11-19 days, 11-18 days, 11-17 days, 11-16 days, 11-15 days, 11-14 days, 11-13 days, 11-12 days, 12-28 days, 12-27 days, 12-26 days, 12-25 days, 12-24 days, 12-23 days, 12-22 days, 12-21 days, 12-20 days, 12-19 days, 12-18 days, 12-17 days, 12-16 days, 12-15 days, 12-14 days, 12-13 days, 13-28 days, 13-27 days, 13-26 days, 13-25 days, 13-24 days, 13-23 days, 13-22 days, 13-21 days, 13-20 days, 13-19 days, 13-18 days, 13-17 days, 13-16 days, 13-15 days, 13-14 days, 14-28 days, 14-27 days, 14-26 days, 14-25 days, 14-24 days, 14-23 days, 14-22 days, 14-21 days, 14-20 days, 14-19 days, 14-18 days, 14-17 days, 14-16 days, 14-15 days, 15-28 days, 15-27 days, 15-26 days, 15-25 days, 15-24 days, 15-23 days, 15-22 days, 15-21 days, 15-20 days, 15-19 days, 15-18 days, 15-17 days, 15-16 days, 16-28 days, 16-27 days, 16-26 days, 16-25 days, 16-24 days, 16-23 days, 16-22 days, 16-21 days, 16-20 days, 16-19 days, 16-18 days, 16-17 days, 17-28 days, 17-27 days, 17-26 days, 17-25 days, 17-24 days, 17-23 days, 17-22 days, 17-21 days, 17-20 days, 17-19 days, 17-18 days, 18-28 days, 18-27 days, 18-26 days, 18-25 days, 18-24 days, 18-23 days, 18-22 days, 18-21 days, 18-20 days, 18-19 days, 19-28 days, 19-27 days, 19-26 days, 19-25 days, 19-24 days, 19-23 days, 19-22 days, 19-21 days, 19-20 days, 20-28 days, 20-27 days, 20-26 days, 20-25 days, 20-24 days, 20-23 days, 20-22 days, 20-21 days, 21-28 days, 21-27 days, 21-26 days, 21-25 days, 21-24 days, 21-23 days, 21-22 days, 22-28 days, 22-27 days, 22-26 days, 22-25 days, 22-24 days, 22-23 days, 23-28 days, 23-27 days, 23-26 days, 23-25 days, 23-24 days, 24-28 days, 24-27 days, 24-26 days, 24-25 days, 25-28 days, 25-27 days, 25-26 days, 26-28 days, 26-27 days, or 27-28 days of age. In some embodiments, the human infant is less than 28 days of age at the time of administration (vaccination). In some embodiments, the human infant is less than 4 days of age at the time of administration (vaccination). In some embodiments, the human infant is less than 2 days of age at the time of administration (vaccination). In some embodiments, the human infant is less than 24 days of age at the time of administration (vaccination). In some embodiments, the administration (vaccination) occurs at birth. In some embodiments, a second administration occurs when the subject is less than or equal to 28 days of age. In some embodiments, a second administration occurs when the subject is less than 6 months of age. In some embodiments, the human subject is a human neonate (e.g., less than 28 days of age) that receives 1 or 2 doses of the vaccine described herein. In some embodiments, the human neonate receives one dose before 28-days of age (e.g., 2, 3, 4, 5, 6, 7, 8, 9, 10, 11, 12, 13, 14, 15, 16, 17, 18, 19, 20 days of age) and a second dose before or at 28-days of age. In some embodiments, a human subject receives one dose at 2 months, 4 months, 6 months, 9 months or 12 months of age, and a second dose after the first dose at 2 months, 4 months, 6 months, 9 months or 12 months of age. In some embodiments, a human subject receives a second dose before or equal to 12-months of age (e.g., 1, 2, 3, 4, 5, 6, 9, or 12 months of age). In some embodiments, a human subject receives a second dose after 6-months of age (e.g., 1 year, 2 years, 3 years of age). In some embodiments, the human subject is born prematurely or has low birth weight. “Born prematurely” means the human subject is born before 40-weeks of term. In some embodiments, the human subject is born before 37-weeks of term. In some embodiments, the human subject is born before 32 weeks of term. In some embodiments, the human subject is born before 24 weeks of term. In some embodiments, the human subject is born before 40 weeks, 39 weeks, 38 weeks, 37 weeks, 36 weeks, 35 weeks, 34 weeks, 33 weeks, 32 weeks, 31 weeks, 30 weeks, 29 weeks, 28 weeks, 27 weeks, 26 weeks, 25 weeks, or 24 weeks of term. In some embodiments, the human subject is more than 28-days old (e.g., 1 month, 2 months, 3 months, 4 months, 5 months, 6 months, 7 months, 8 months, 9 months, 10 months, 11 months, 12 months, 2 years, 3 years, 4 years, 5 years, 10 years, 11 years, 12 years, 13 years, 14 years, 15 years, 16 years, 17 years old). In some embodiments, the human subject is born with low birth weight (e.g., at least 20% lower than a normal birth weight). In some embodiments, the human subject has an undeveloped (e.g., an infant or a neonate), weak (an older individual), or compromised immune system. Immunocompromised subjects include, without limitation, subjects suffering from sepsis, HIV patients, and patients who received radiation (e.g., for the treatment of cancer). In some embodiments, the human subject is a pediatric human (e.g., up to 18 years old). In some embodiments, the human subject is an adult (e.g., more than 18 years old). In some embodiments, the human subject is an older human (e.g., more than 60 years old, or 65 years of age or older, or more than 65 years of age). In some embodiments, the human neonate is 1, 2, 3, 4, 5, 6, 7, 8, 9, 10, 11, 12, 13, 14, 15, 16, 17, 18, 19, 20, 21, 22, 23, 24, 25, 26, 27, or 28 days of age at the time of administration of the compound described herein. In some embodiments, the human subject is an infant. In some embodiments, the human subject is a pediatric human of up to 18 years of age. In certain embodiments, the subject is an adult human (i.e., 18 years of age or older). In certain embodiments, the subject is an older adult human. In certain embodiments, the subject is 65 years of age or older. In certain embodiments, the subject is more than 65 years of age. In some embodiments, the subject is immunocompromised (e.g., due to primary immunodeficiency, acquired immunodeficiency, and/or distinct immunity). In some embodiments, the subject has primary or acquired immunodeficiency or demonstrates a distinct immune profile due to demographic features and/or co-morbidities. In some embodiments, the human subject receives one or two doses of the vaccine described herein after 65-years of age. In certain embodiments, the subject is a non-human animal. In certain embodiments, the cell is in vitro. In certain embodiments, the cell is in vivo.

In another aspect, the present disclosure provides Compound (1) for use in a method described herein (e.g., a method of enhancing an immune response (e.g., innate and/or adaptive immune response), a method of treating a disease (e.g., a proliferative disease, an inflammatory disease, an autoimmune disease, an infectious disease, an allergy, a fibrotic disease, a cardiovascular disease, a graft rejection, graft-versus-host disease, chronic disease, or addiction), and a method of treating and/or preventing a disease by using Compound (1) as a medicament or adjuvant in a vaccine for the disease (e.g., a proliferative disease, an inflammatory disease, an autoimmune disease, an infectious disease, an allergy, a fibrotic disease, a cardiovascular disease, a graft rejection, graft-versus-host disease, chronic disease, or addiction), or as stand alone anti-infective or immune response modifying agent. In certain embodiments, the present disclosure provides Compound (1) for use as an immunomodulator.

In another aspect, the present disclosure provides Compound (1) for use in treating and/or preventing a proliferative disease in a subject. In another aspect, the present disclosure provides Compound (1) for use as a medicament.

In still another aspect, the present disclosure provides the pharmaceutical compositions described herein for use in a method described herein (e.g., a method of enhancing an immune response (e.g., innate and/or adaptive immune response), a method of treating a disease (e.g., a proliferative disease, an inflammatory disease, an autoimmune disease, an infectious disease, an allergy, a fibrotic disease, a cardiovascular disease, a graft rejection, graft-versus-host disease, chronic disease, or addiction), and a method of treating and/or preventing a disease by using Compound (1) as a medicament or adjuvant in a vaccine for the disease (e.g., a proliferative disease, an inflammatory disease, an autoimmune disease, an infectious disease, an allergy, a fibrotic disease, a cardiovascular disease, a graft rejection, graft-versus-host disease, chronic disease, or addiction), or as stand alone anti-infective or immune response modifying agent.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting their scope.

### Preparation of the Compounds Described Herein

Compound (1) can be prepared from readily available starting materials using the following scheme and according to the procedures in US 2022/0242867, incorporated herein by reference. Where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

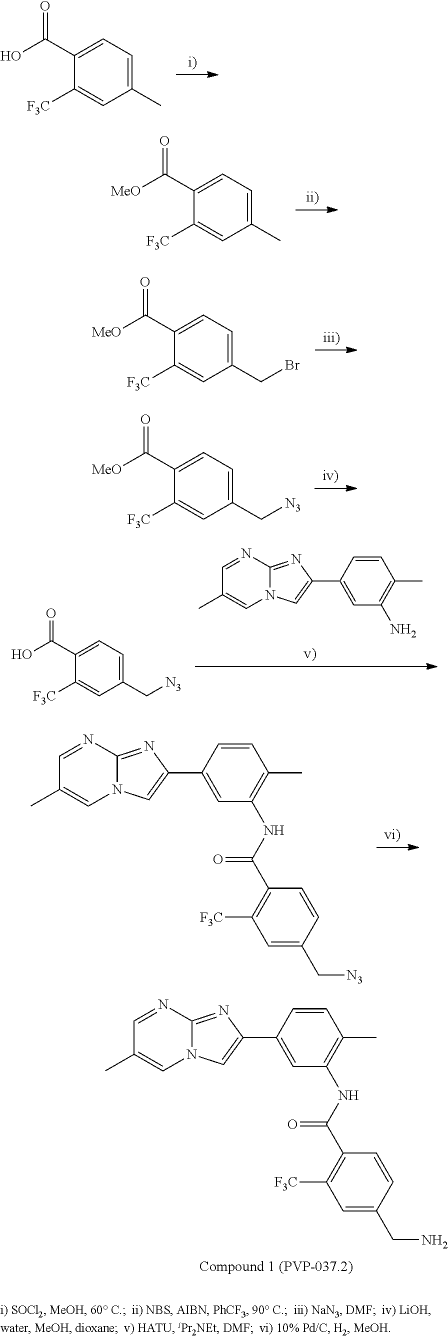

### Methyl 4-methyl-2-(trifluoromethyl) benzoate

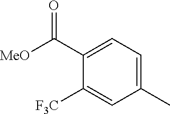

Thionyl chloride (5.25 g 3.2 ml, 0.44 Mol) was added dropwise over 15 mins to a cooled solution (0° C.) of 4-methyl-2-(trifluoromethyl) benzoic acid 3.0 g (14.7 mmol) in MeOH (60 mL). The solution was stirred at RT for 1 hour, then at 60° C. overnight. The excess thionyl chloride and MeOH was removed under reduced pressure. The residue was partitioned between between EtOAc (100 mL) and brine (100 mL) and sat. aq. NaHCO₃ solution was added to adjust the pH to 8. The organic layer was washed with brine, dried over Na₂SO₄, filtered and concentrated to give the title compound (2.63 g, yield 82%). MS m/z 219.18 [M+H]⁺.

### Methyl 4-(bromomethyl)-2-(trifluoromethyl) benzoate

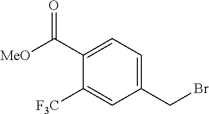 [SMILES: COC(=O)c1ccc(CBr)cc1C(F)(F)F]

N-Bromosuccinimide (2.48 g, 14.0 mmol) and AIBN (160 mg, 0.97 mmol) were added to a solution of methyl 4-methyl-2-(trifluoromethyl) benzoate (2.90 g, 13.3 mmol) in trifluorotoluene (30 mL). The reaction mixture was stirred at 90° C. for 2 hours, then allowed to cool. EtOAc (100 mL) and 1N HCl (100 mL) were added, and the organic layer was washed twice with brine, dried over Na₂SO₄, filtered and concentrated. The residue was purified by silica chromatography (9:1 Hex:EtOAc) to give the title compound (1.89 g 48%). ¹H NMR (500 MHz, CDCl₃-d): δ 7.80-7.54 (m, 2H) 7.68-7.60 (m, 1H), 4.51 (s, 2H) 3.95 (s, 3H). MS m/z 298.23 [M+H]⁺.

### Methyl 4-(azidomethyl)-2-(trifluoromethyl) benzoate

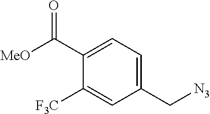 [SMILES: COC(=O)c1ccc(CN=[N+]=[N-])cc1C(F)(F)F]

Sodium azide (497 mg, 7.65 mmol) was added to a solution of methyl 4-(bromomethyl)-2-(trifluoromethyl) benzoate (1.89 g, 6.37 mmol) in DMF (20 mL). The reaction mixture was stirred at RT under nitrogen for 12 hours. EtOAc (100 mL) and brine (100 mL) were added, and the organic layer was washed twice with brine, dried over Na₂SO₄, filtered and concentrated to give the title compound (1.65 g 100%). ¹H NMR (500 MHz, CDCl₃-d): δ 7.85-7.79 (m, 1H) 7.70 (m, 1H) 7.60-7.54 (m, 1H) 4.50 (s, 2H) 3.95 (s, 3H).

### 4-(Azidomethyl)-2-(trifluoromethyl) benzoic acid

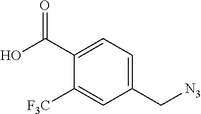 [SMILES: [N-]=[N+]=NCc1ccc(C(=O)O)c(C(F)(F)F)c1]

Methyl 4-(azidomethyl)-2-(trifluoromethyl) benzoate (500 mg, 1.93 mmol) was dissolved in dioxane (30 mL). MeOH (3 mL), water (3 mL) and LiOH·H₂O (243 mg, 579 mMol) were added to the solution. The mixture was stirred at RT for 12 hours. EtOAc (100 mL) and brine (100 mL) were added to the reaction mixture, and the pH was adjusted to 5 with 1N HCl. The organic layer was washed twice with brine, dried over Na₂SO₄, filtered and concentrated to give the title compound (473 mg 100%) and used without further purification.

### 2-Methyl-5-(6-methylimidazo[1,2-a]pyrimidin-2-yl)aniline

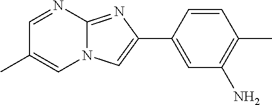

This compound was prepared as described in WO2019099564.

### 4-(Azidomethyl)-N-(2-methyl-5-(6-methylimidazo[1,2-a]pyrimidin-2-yl) phenyl)-2-(trifluoromethyl) benzamide

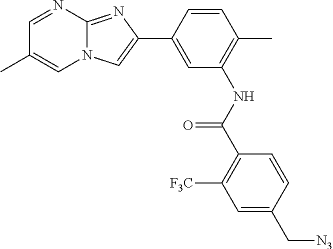 [SMILES: Cc1cnc2nc(-c3ccc(C)c(NC(=O)c4ccc(CN=[N+]=[N-])cc4C(F)(F)F)c3)cn2c1]

4-(Azidomethyl)-2-(trifluoromethyl)benzoic acid (473 mg, 1.93 mmol), HATU (1.83 g, 4.82 mmol) and DIEA (1.04 g, 1.4 mL, 8.04 mmol) were added to a solution of 2-methyl-5-(6-methylimidazo[1,2-a]pyrimidin-2-yl)aniline (383 mg, 1.61 mmol) in DMF (5 mL), and the mixture was stirred at RT for 12 hours. EtOAc (100 mL) and brine (100 mL) were added, and the organic layer was washed twice with brine, dried over Na₂SO₄, filtered and concentrated. The residue was purified by silica chromatography (5% MeOH in DCM) to give the title compound (334 mg, 45%). ¹H NMR (500 MHz, DMSO-d6): δ 10.15 (s, 1H), 8.79-8.73 (m, 1H), 8.46-8.40 (m, 1H), 8.30, 8.24 (m, 1H), 8.1-8.05 (m, 1H), 7.9-7.74 (m, 4H), 7.4-7.32 (m, 1H), 4.68 (s, 2H), 2.34 (d, 6H). MS m/z 466.27 [M+H]⁺.

### Compound 1 (PVP-037.2): 4-(Aminomethyl)-N-(2-methyl-5-(6-methylimidazo[1,2-a]pyrimidin-2-yl) phenyl)-2-(trifluoromethyl) benzamide

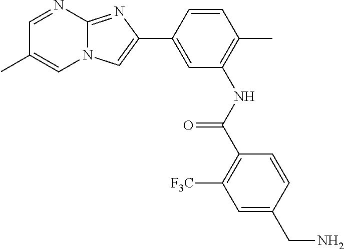 [SMILES: Cc1cnc2nc(-c3ccc(C)c(NC(=O)c4ccc(CN)cc4C(F)(F)F)c3)cn2c1]

Pd/C (30 mg, 10% w/w) was added to a solution of 4-(azidomethyl)-N-(2-methyl-5-(6-methylimidazo[1,2-a]pyrimidin-2-yl) phenyl)-2-(trifluoromethyl) benzamide (300 mg, 0.644 mmol) in MeOH (100 mL), and the reaction vessel was evaluated to hydrogen gas. The mixture was stirred at for 12 hours, filtered through Celite, and concentrated under reduced pressure to give the title compound (220 mg, 78%). ¹H NMR (500 MHz, DMSO-d6): δ 10.1 (s, 1H), 8.8-8.7 (m, 1H), 8.46-8.35 (m, 1H), 8.3-8.22 (m, 1H), 8.1-8.0 (m, 1H), 7.9-7.86 (m, 1H), 7.8-7.72 (m, 3H), 7.39-7.32 (m, 1H), 3.97 (s, 2H), 2.33 (d, 6H). MS m/z 439.86 [M+H]⁺.

### Compound (1) has Improved Solubility, Enhanced In Vitro Potency and In Vivo Immunogenicity Over PVP-037.1

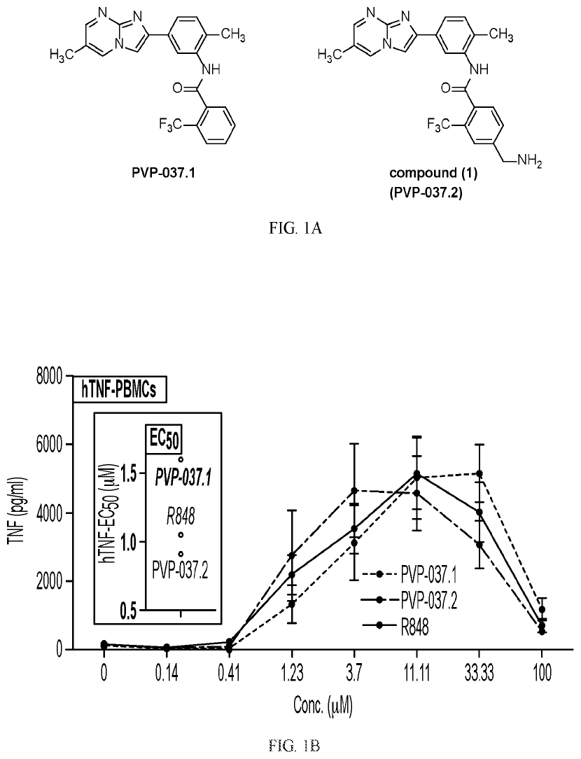

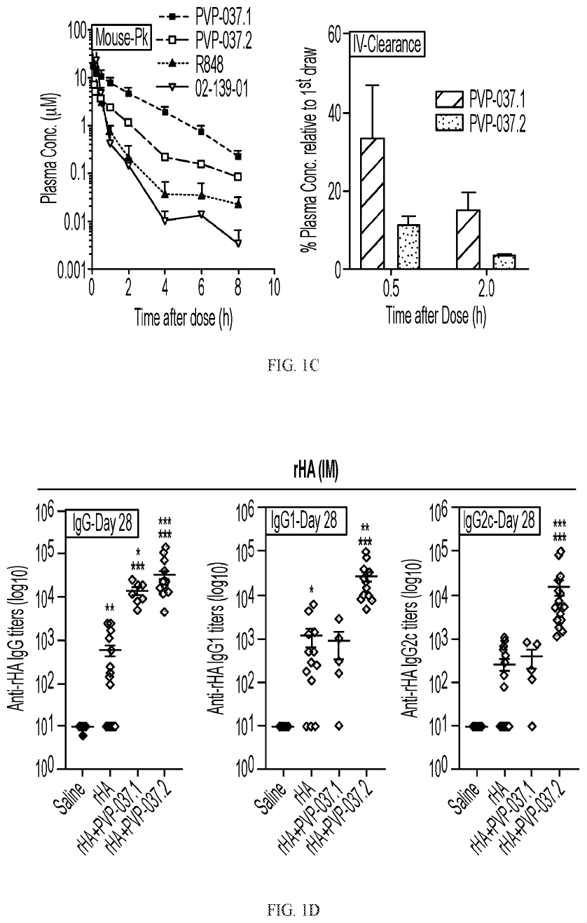

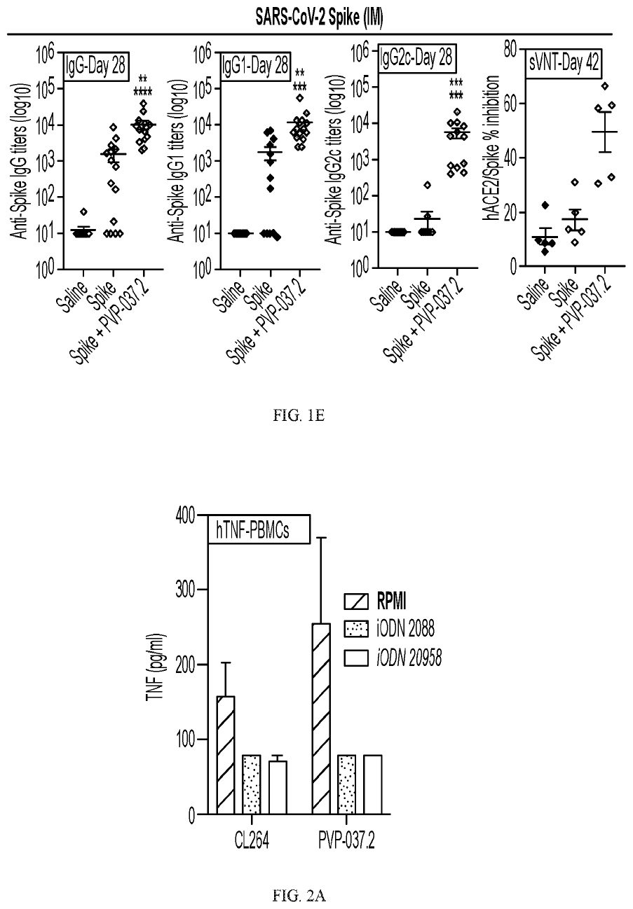

Compound (1) demonstrated improved solubility. Upon evaluation via human PBMC in vitro assays, Compound (1) had greater TNF-inducing efficacy, and strikingly, Compound (1) had a lower EC₅₀ compared to PVP-037.1 and R848. (FIG. 1B). In addition, Compound (1) has an improved in vivo profile. Mouse pharmacokinetic evaluation of drug serum concentrations at 8-hours post-administration demonstrated that relative to PVP-037.1, Compound (1) had enhanced clearance from blood plasma and negligible hemolytic activity (FIG. 1C).

The ability of the compound to enhance antigen immunogenicity in vivo was evaluated. Adult (~6-8-weeks-old) C57BL/6 mice IM were immunized using a prime-only schedule (Day 28) and employing trivalent recombinant hemagglutinin (rHA) influenza vaccine Flublok as a clinically relevant model antigen that is devoid of adjuvant, alone or formulated with PVP-037.1 or Compound (1) (100 nmol/mouse), and Ab titers for rHA-specific IgG were measured by ELISA 28 days post-immunization (FIG. 1D). The titers of the IgG subclasses IgG1 and IgG2c were also investigated, respectively associated with type 2 and type 1 (IFNγ-driven) immunity. Both PVP-037.1 and Compound (1) increased anti-rHA IgG titers in adult mice. Interestingly and in line with in vitro observations, only Compound (1) was able to, via a single immunization, significantly enhance antigen-specific IgG1 and IgG2c production (FIG. 1D).

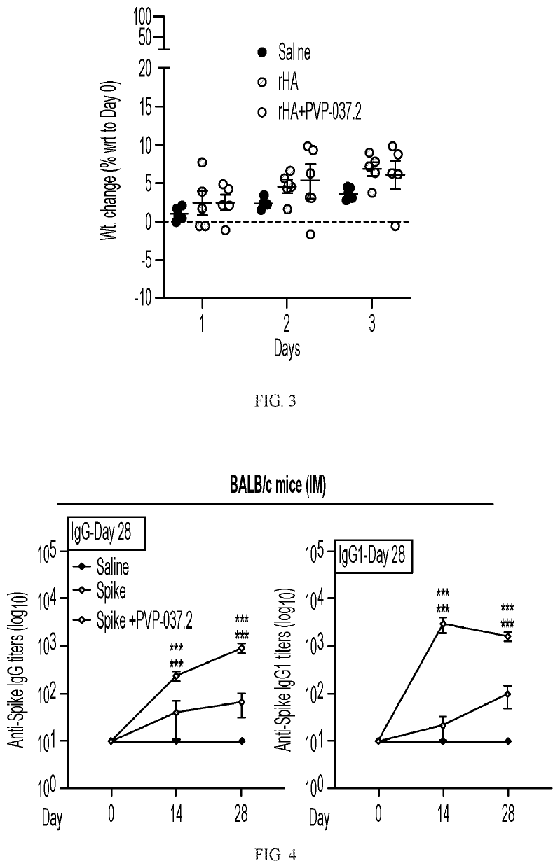

To characterize the adjuvanticity of Compound (1), a secondary antigen and strain of mouse were evaluated. C57BL/6 adult mice were injected IM prime (Day 0) with saline, WT SARS-CoV-2 spike protein alone, spike admixed with Compound (1) (100 nmoles/mouse). Ab titers for WT spike-specific IgG, IgG1 or IgG2c were measured by ELISA 28 days post-immunization. Again, Compound (1) via a single immunization, drove enhancement of IgG, IgG1 and IgG2c. Young mice (6-8 weeks old) vaccinated with Compound (1) maintained normal weight gains (FIG. 3). Additionally, Compound (1) demonstrated robust adjuvanticity across mouse strains (FIG. 4). Via a prime-boost (Day 28) schedule, Compound (1) hACE2-RBD (WT) inhibition was determined 42 days post-immunization (FIG. 1E). Overall, these results support robust immunogenicity enhancement via Compound (1) as an adjuvant for multiple routes and antigens.

### Compound (1) Demonstrates a TLR7-Dependent Immunomodulatory Profile

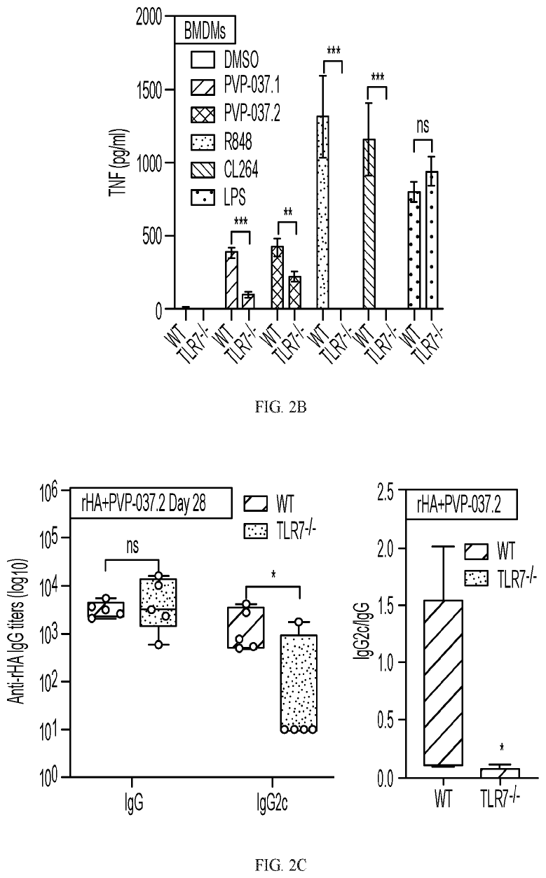

Initial in vitro evaluation of imidazopyrimidine compounds via human embryonic kidney (HEK)293 cells expressing human pattern recognition receptors (PRRs), including both HEK293-hTLR7 and hTLR8 NFκB-SEAP reporter cells from Novus Biologicals (Littleton, CO) and InvivoGen (San Diego, CA) respectively, demonstrated minimal activity (unpublished observations). However, since a) endosomal receptors are disproportionately the target for small molecule agonists adjuvants, and b) published reports have outlined difficulties reproducing TLR7/8 specificity using HEK293-hTLR7 cells, the activity of Compound (1) in PBMC was investigated after treatment with the short single-stranded oligodeoxynucleotides (ODN) 2088, a known TLR7/8/9 antagonist. Here, a near total loss of activity of Compound (1) was observed (FIG. 2A). Furthermore, in human PBMCs, ODN 20958 (TLR7 specific antagonist) completely blocked Compound (1)-induced TNF production, that mirrored the response of CL264, a highly selective TLR7A. In contrast, inhibition of TLR8 using CU-CPT9a (which is a selective inhibitor for TLR8) did not affect Compound (1)-induced cytokine production in human PBMCs (not shown). To confirm this observation, the mechanistic pathways induced by PVP-037 analogs utilizing bone marrow derived macrophages (BMDM) from both WT and TLR7^{-/-} mice was investigated. Here, both PVP-037.1 and Compound (1) induced TNF production, that was significantly reduced in BMDM from TLR7^{-/-} mice as compared to both CL264 and R848. In contrast, responses to LPS were unaffected in TLR7^{-/-} BMDMs (FIG. 2B). Based on these in vitro observations, the in vivo adjuvanticity of a single dose of Compound (1) towards rHA using WT and TLR7^{-/-} adult mice was evaluated. Ab titers for rHA-specific IgG or IgG2c were measured at 28 days post-immunization. A significant reduction in IgG2c class switched Ab response was observed in the TLR7^{-/-} mice (FIG. 2C), strongly suggesting that the type 1 immunomodulatory profile of Compound (1) is mediated by TLR7.

### Materials and Methods

Collection of human blood, isolation and in vitro assay of PBMCs. Peripheral blood was collected from healthy adult volunteers. Human experimentation guidelines of the U.S. Department of Health and Human Services and Boston, and Boston Children's Hospital were observed, following protocols approved by the local institutional review boards. Human blood was anti-coagulated with 20 units/ml pyrogen-free sodium heparin (American Pharmaceutical Partners, Inc.; Schaumberg, IL). All blood products were kept at room temperature and processed within 4 hrs from collection as previously described (D. J. Dowling et al., PLoS One 8, e58164 (2013); D. J. Dowling et al., JCI Insight, 2, e91020 (2017)). PBMCs were isolated from blood using a Ficoll density gradient. PBMCs either used fresh or were stored at 5×10⁷ cells per vial in 1 mL RPMI containing 20% autologous plasma and 10% DMSO at −80° C. until use. Stimulation plates were prepared by transferring 0.66 μl of DMSO-dissolved compounds (10 mM) to each well of a round bottom 96-well plate. PBMCs isolated from human adult donors were resuspended at a concentration of 10⁵ cells/200 μl of RPMI supplemented with 10% of platelet-poor plasma. 200 μl of the cell suspension were transferred to each well resulting in a final compound concentration (e.g., 33 M). After −18 hrs of incubation (37° C., 5% CO₂), plates were centrifuged (500×g, room temperature, 5 minutes) and supernatants were harvested for further analysis.

ELISAs and Multiplex-Analyte Assays. Supernatants derived from human PBMCs and DC stimulations were assayed by ELISA for TNF (ThermoFisher Scientific; Waltham, MA, USA). Cytokine and chemokine expression profiles (e.g., IFNγ, IL-9, IL-10, IL-12 (p70), IL-13, IL-1β, IL-23, IL-27, IL-28A, IL-33, IL-6, CCL20 (aka MIP-3α), and TNF) in cell culture supernatants were measured using a customized Milliplex Human Th17 Magnetic Bead Panel according to the manufacturer's instructions (Millipore; Chicago, IL, USA). Assays were read and analyzed on the Luminex 100/200 System and xPOTENT software (Luminex; Austin, TX). A minimum threshold was set at the minimum detectable concentration for each individual assay, defined as three standard deviations above the mean background. Supernatants derived from murine splenocytes and macrophage stimulations were assayed by ELISA for TNF and/or IL-6 (ThermoFisher Scientific).

Endotoxin measurement and human TLR7- and TLR8-transfected HEK293 assay. All TLR7/8As in both in vitro and in vivo studies were verified to be free of endotoxin (<1 EU/ml) by the Endosafe nexgen-MCS Limulus amoebocyte lysate assay per the manufacturer's instructions (Charles River Laboratories). Human embryonic kidney (HEK)293 cells expressing human TLR7 or TLR8 with an NF-κB-responsive secreted embryonic alkaline phosphatase (SEAP) reporter gene were obtained from Novus Biologicals (Littleton, CO) and InvivoGen (San Diego, CA), respectively. Cells were maintained in DMEM with 10% heat inactivated-FBS and selection antibiotics per the manufacturer's instructions. Cells were plated at 5×10⁵ cells/96-well and stimulated with indicated agonist(s) for 24 h. Supernatants were harvested and analyzed for NF-κB/SEAP activation using the QuantiBlue kit (InvivoGen). Values are expressed as fold change in OD₆₅₀ over vehicle-only treated samples.

Human MoDCs and Mouse Splenocytes and Macrophages. Monocytes were isolated from PBMC fractions by positive selection by magnetic microbeads according to the manufacturer's instructions (Miltenyi Biotec, Auburn, CA) using CD14 as a pan marker. Isolated monocytes were cultured in tissue culture dishes at 10⁶ cells/ml in RPMI 1640 media containing fresh 10% autologous plasma, supplemented with recombinant human IL-4 (50 ng/ml) and recombinant human GM-CSF (100 ng/ml) (R&D Systems, Minneapolis, MN) with one additional supplement of fresh media and cytokines at day 3 of culture as previously described (D. J. Dowling et al., J Allergy Clin Immunol 140, 1339-1350 (2017); L. Ganapathi et al., PLoS One 10, e0134640 (2015)). After 6 days, immature MoDCs were harvested by gently pipetting the loosely adherent fraction, before being re-plated (10⁵ cells/well) in 96-well U-bottom plates in the presence or absence of treatments and/or sterile PBS. Plates were then incubated for 18-24 hrs at 37° C. in a humidified incubator at 5% CO₂. After this stimulation supernatants were harvested and processed for further functional assays. To isolate murine splenocytes, spleens were mashed through a 70 m cell strainer, and the resulting cell suspensions were washed with PBS and incubated with 2 mL of ACK lysis buffer (Gibco) for 2 min at room temperature to lyse erythrocytes. Splenocytes were washed again with PBS and plated in flat-bottom 96-well plates (2×10⁶ cells per well) for stimulation for 48 hrs, supernatants were harvested, and cytokine concentrations were measured by ELISA (E. Nanishi et al., Sci Transl Med 14, eabj5305 (2022)). Mouse macrophages were derived from isolated bone marrow-derived progenitor cells via their propagation in M-CSF-containing medium. After 7 days in culture, with one additional supplement of fresh media and cytokines at day 3 of culture, contaminating nonadherent cells are eliminated by gently pipetting the loosely adherent fraction, adherent cells are harvested for assays, before being re-plated (10⁵ cells/well) in 96-well U-bottom plates in the presence or absence of treatments and/or sterile PBS. Adherent bone marrow-derived macrophages are routinely >90% pure (X. Zhang, R. Goncalves, D. M. Mosser, The isolation and characterization of murine macrophages. Current protocols in immunology Chapter 14, Unit 14.11 (2008)).

Influenza antigens, vaccination and antibody quantification. For vaccination experiments, adult mice, typically 6-8 weeks of age, were immunized intramuscularly (IM) in the right posterior thigh with 50 μl of vaccine dose containing 0.25 μg of each of the following recombinant influenza virus hemagglutinins (rHA): A/Michigan/45/2015 (H1N1), A/Singapore/INFIMH-16-0019/2016 (H3N2), B/Maryland/15/2016 (a B/Colorado/6/2017-like virus), and B/Phuket/3073/2013, derived from seasonal FluBlok vaccine (Protein Sciences Corp.). Mice were vaccinated with a prime-boost schedule (two injections) four weeks apart. Each vaccine dose was formulated with 10% (v/v) DMSO. As indicated for specific experimental groups, each dose may have been also formulated with aluminium hydroxide (100 g) and/or a compound described herein (each compound at 100 nmol, final DMSO concentration 10%). Serum was collected 28 days post-prime (pre-boost blood sample) and 14 days post-boost for antibody detection. rHA-specific IgG were quantified by ELISA. High binding flat bottom 96-well plates (Corning Life Sciences) were coated with 1 μg/ml rHA in carbonate buffer pH 9.6, incubated overnight at 4° C. and blocked with PBS+BSA 1% (Sigma-Aldrich) for 1 h at room temperature (RT). Then, sera from vaccinated mice were added with an initial dilution of 1:100 and 1:4 serial dilutions in PBS+BSA 1% and incubated for 2 hrs at RT. Plates were then washed and incubated for 1 hrs at RT with HRP-conjugated anti-mouse IgG (Southern Biotech). At the end of the incubation plates were washed again and developed with tetramethylbenzidine (BD Biosciences) for 5 minutes, then stopped with 1 N H₂SO₄. The optical density was read at 450 nm Versamax microplate reader with SoftMax Pro Version 5 (both from Molecular Devices) and endpoint titers were calculated using as cutoff three times the optical density of the background.

SARS-CoV-2 wild-type spike and RBD expression/purification and serology. Full length SARS-CoV-2 Wuhan-Hu-1 spike glycoprotein (M1-Q1208, GenBank MN90894) with an HRV3C protease cleavage site, a TwinStrepTag and an 8×HisTag at C-terminus were obtained from Barney S. Graham (NIH Vaccine Research Center). These mammalian expression vectors were used to transfect Expi293F suspension cells (Thermo Fisher) using polyethylenimine (Polysciences). Cells were allowed to grow in 37° C., 8% CO₂ for an additional 5 days before harvesting for purification. Protein was purified in a PBS buffer (pH 7.4) from filtered supernatants by using either StrepTactin resin (IBA) or Cobalt-TALON resin (Takara). Affinity tags were cleaved off from eluted protein samples by HRV 3C protease, and tag removed proteins were further purified by size-exclusion chromatography using a Superose 6 10/300 column (Cytiva) for full length Spike in a PBS buffer (pH 7.4).

Mice were vaccinated as above (1 μg of Spike Protein per mouse). Spike protein-specific antibody concentrations were quantified in serum samples by ELISA by modification of a previously described protocol (F. Borriello et al., Frontiers in immunology 8, 1772 (2017)). Briefly, high-binding flat-bottom 96-well plates (Corning) were coated with spike protein (25 ng per well) and incubated overnight at 4° C. Plates were washed with 0.05% Tween 20/PBS and blocked with 1% bovine serum albumin (BSA)/PBS for 1 hour at room temperature. Serum samples were serially diluted fourfold from 1:100 up to 1:1.05×10⁸ and then incubated for 2 hours at room temperature. Plates were washed three times and incubated for 1 hour at room temperature with horseradish peroxidase (HRP)-conjugated anti-mouse IgG, IgG1, and IgG2a (SouthernBiotech). Plates were washed five times and developed with tetramethylbenzidine (BD OptEIA Substrate Solution, BD Biosciences) for 5 min and then stopped with 2 N H2SO4. Optical densities (ODs) were read at 450 nm with a SpectraMax iD3 microplate reader (Molecular Devices). Endpoint titers were calculated as the dilution that emitted an OD exceeding a 3× background. An arbitrary value of 50 was assigned to the samples with OD values below the limit of detection for which it was not possible to interpolate the titer.

The hACE2-RBD inhibition assay used a modification of a previously published protocol (C. W. Tan et al., Nat Biotechnol 38, 1073-1078 (2020)). Briefly, high-binding flat-bottom 96-well plates (Corning) were coated with recombinant hACE2 (100 ng per well) (Sigma-Aldrich) in PBS, incubated overnight at 4° C., washed three times with 0.05% Tween 20 PBS, and blocked with 1% BSA PBS for 1 hour at RT. Serum samples were diluted 1:160, preincubated with 3 ng of wild-type RBD-Fc in 1% BSA PBS for 1 hour at RT, and then transferred to the hACE2-coated plate. RBD-Fc without preincubation with serum samples was added as a positive control, and 1% BSA PBS without serum preincubation was added as a negative control. Plates were then washed three times and incubated with HRP-conjugated anti-human IgG Fc (SouthernBiotech) for 1 hour at RT. Plates were washed five times and developed with tetramethylbenzidine (BD OptEIA Substrate Solution, BD Biosciences) for 5 min and then stopped with 2 N H₂SO₄. The OD was read at 450 nm with a SpectraMax iD3 microplate reader (Molecular Devices). Percentage inhibition of RBD binding to hACE2 was calculated as: Inhibition (%)=[1−(Sample OD value−Negative Control OD value)/(Positive Control OD value−Negative Control OD value)]×100.

Statistical analyses and graphics. Statistical significance and graphic outputs were generated using GraphPad Prism version 8 for macOS (GraphPad Software; La Jolla, CA, USA) and Microsoft Excel (Microsoft Corporation; Redmond, WA). For non-HTS in vitro and in vivo experiments where values were normalized to control, column statistics were conducted using two-tailed Wilcoxon Signed Rank Test or one-sample T test comparing to control. Group comparisons were performed by One-way ANOVA with Dunnett's multiple comparison post-test or Two-way ANOVA comparing column and row effects. Results were considered significant at p<0.05, and indicated as follows: *p<0.05, **p<0.01, ***p<0.001, ****p<0.0001. Graphics were created with BioRender.com.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound of formula: 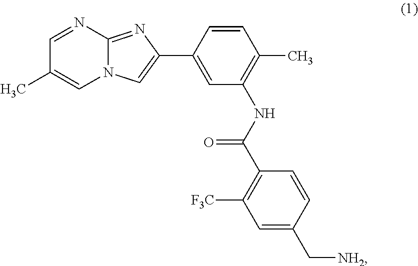 [SMILES: Cc1cnc2nc(-c3ccc(C)c(NC(=O)c4ccc(CN)cc4C(F)(F)F)c3)cn2c1]
or a pharmaceutically acceptable salt, solvate, hydrate, polymorph, co-crystal, tautomer, stereoisomer, isotopically labeled derivative, or prodrug thereof.

2. The compound of claim 1, or a pharmaceutically acceptable salt thereof.

3. A compound of formula:

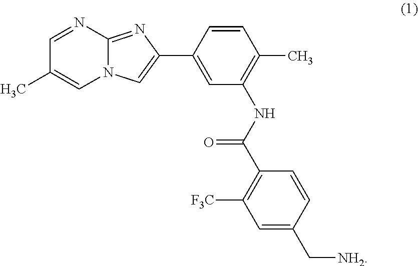 [SMILES: Cc1cnc2nc(-c3ccc(C)c(NC(=O)c4ccc(CN)cc4C(F)(F)F)c3)cn2c1]

4. A pharmaceutical composition comprising the compound of any one of claims 1-3, and a pharmaceutically acceptable excipient.

5. The pharmaceutical composition of claim 4 further comprising an additional pharmaceutical agent.

6. A method of treating a disease in a subject in need thereof, the method comprising administering the compound of any of claims 1-3, the pharmaceutical composition of claim 4 or 5, the composition of any of claims 40-63, or the vaccine of any of claims 64-67.

7. The method of claim 6, wherein the disease is a proliferative disease, an inflammatory disease, an autoimmune disease, an infectious disease, an allergy, a fibrotic disease, a cardiovascular disease, a graft rejection, graft-versus-host disease, chronic disease, or addiction.

8. The method of claim 6 or 7, wherein the disease is an infectious disease such as a viral infectious disease, sepsis, a pediatric infectious disease, a bacterial infectious disease, a mycobacterial infectious disease, a parasitic infectious disease, or a fungal infectious disease.

9. The method of any of claims 6-8, wherein the disease is a bacterial infectious disease caused by infection with Streptococcus spp., Bordetella spp., Haemophilus influenzae type b, Neisseria spp., Vibrio spp., Yersinia spp., Staphylococci spp., or Salmonella spp.

10. The method of any of claims 6-8, wherein the disease is a bacterial infectious disease that is anthrax, diphtheria, tetanus, or cholera.

11. The method of any of claims 6-8, wherein the disease is a mycobacterial infectious disease caused by a tuberculosis infection or a non-tuberculous mycobacterial infection.

12. The method of any of claims 6-8, wherein the disease is a viral infectious disease caused by adenovirus, polio virus, Ebola, herpes viruses (e.g., herpes simplex virus 1, herpes simplex virus 2, human herpesvirus 6, human herpesvirus 8), cytomegalovirus, varicella-zoster virus, measles, mumps, rubella, hepatitis A, hepatitis B, hepatitis C, human papilloma virus, influenza, parainfluenza virus, coronavirus (e.g., SARS-CoV-2), rabies, Japanese encephalitis, rotavirus, human immunodeficiency virus, respiratory syncytial virus, monkeypox, smallpox, yellow fever, or Zika Virus.

13. The method of any of claims 6-8, wherein the disease is a parasitic infectious disease that is malaria or leishmaniasis.

14. The method of claim 6 or 7, wherein the disease is cancer.

15. The method of any of claims 6-8, wherein the disease is a fungal infectious disease caused by Candida spp., Aspergillus spp., Cryptococcus spp., Mucoromycete, Blastomyces dermatitidis, Histoplasma capsulatum, or Sporothrix schenckii.

16. The method of any of claims 6-15, wherein the composition is administered once to the subject.

17. The method of any of claims 6-15, wherein the composition is administered repeatedly to the subject.

18. The method of any of claims 6-17, wherein the composition is administered intradermally, intramuscularly, intravaginally, intravenously, intranasally, orally, subcutaneously, transdermally, topically, and/or sublingually.

19. The method of any of claims 6-18, wherein the composition is administered as a prophylactic.

20. The method of any of claims 6-19, wherein the composition is administered as a combination therapy with another immunomodulatory agent, an immunomodulating antibody, an immunomodulating biologic, or an inhibitor of molecular pathways that limits immune responses.

21. The method of any of claims 6-20, wherein the subject is a human infant.

22. The method of claim 11, wherein the human infant is less than or equal to 28 days of age, less than or equal to 4 days of age, less than or equal to 2 days of age, or less than or equal to 24 hours of age at the time of administration, or the administration occurs at birth.

23. The method of claim 22, wherein a second administration occurs when the human infant is less than or equal to 28 days of age, or less than or equal to 6 months of age.

24. The method of claim 21, wherein the administration occurs when the human infant is 2 months, 4 months, and 6 months of age.

25. The method of any of claims 21-24, wherein the infant is born prematurely or has low birth weight.

26. The method of any of claims 6-20, wherein the subject is a pediatric human of up to 18 years of age.

27. The method of any of claims 6-20, wherein the subject is a human adult.

28. The method of any of claims 6-20, wherein the subject is a human adult of 65 years of age or older.

29. The method of any of claims 6-28, wherein the subject is immunocompromised due to primary immunodeficiency, acquired immunodeficiency and/or distinct immunity.

30. A method of enhancing an immune response in a subject in need thereof, the method comprising administering to the subject a therapeutically effective amount of the compound of any of claims 1-3, the pharmaceutical composition of claim 4 or 5, the composition of any of claims 40-63, or the vaccine of any of claims 64-67.

31. The method of claim 30, wherein the immune response is an innate immune response.

32. The method of claim 30 or 31, wherein the compound activates peripheral blood mononuclear cells (PBMCs).

33. The method of any of claims 30-32, wherein the compound activates one or more pattern recognition receptors (PRRs).

34. The method of claim 33, wherein the PRR is a Toll-like receptor (TLR), a NOD-like receptor (NLR), a RIG-I-like receptor, a C-type Lectin receptor, or STING.

35. The method of claim 34, wherein the Toll-like receptor is TLR1, TLR2, TLR3, TLR4, TLR5, TLR6, TLR9, and/or TLR10.

36. The method of claim 34, wherein the Toll-like receptor is TLR7 and/or TLR8.

37. The method of any of claims 30-36, wherein the compound induces the production of immunomodulatory cytokines and/or chemokines in the subject.

38. The method of claim 35, wherein the cytokines and chemokines include TNF, IL-12, IL-6, IL-10, or CXCL-8.

39. A kit comprising the compound of any of claims 1-3, the pharmaceutical composition of claim 4 or 5, the composition of any of claims 40-63, or the vaccine of any of claims 64-67; and instructions for use.

40. A composition comprising an antigen and the compound of claim 1, or a pharmaceutically acceptable salt, solvate, hydrate, polymorph, co-crystal, tautomer, stereoisomer, isotopically labeled derivative, or prodrug thereof.

41. The composition of claim 40, wherein the antigen comprises a protein or polypeptide.

42. The composition of claim 40 or 41, wherein the antigen comprises a nucleic acid encoding a protein or a polypeptide.

43. The composition of claim 42, wherein the nucleic acid is DNA or RNA.

44. The composition of any of claims 40-43, wherein the antigen is from a microbial pathogen.

45. The composition of claim 45, wherein the microbial pathogen is a bacterium, mycobacterium, fungus, virus, parasite, or prion.

46. The composition of claim 45, wherein the microbial pathogen is a bacterium selected from the group consisting of Bacillus spp., Bordetella spp., Corynebacterium spp, Clostridium spp, Haemophilus spp., Streptococcus spp, Staphylococci spp., Mycobacterium spp., Neisseria spp., Salmonella spp., Vibrio spp., and Yersinia spp.

47. The composition of claim 45, wherein the microbial pathogen is a virus selected from the group consisting of adenovirus, enterovirus (e.g., polio virus), Ebola virus, herpes viruses (e.g., herpes simplex virus 1, herpes simplex virus 2, human herpesvirus 6, human herpesvirus 8), cytomegalovirus, varicella-zoster, measles, mumps, rubella, hepatitis A virus, hepatitis B virus, hepatitis C virus, human papilloma virus, influenza virus, parainfluenza virus, coronavirus (e.g., SARS-CoV-2), rabies, Japanese encephalitis, rotavirus, human immunodeficiency virus (HIV), respiratory syncytial virus (RSV), smallpox, monkeypox, yellow fever, and Zika Virus.

48. The composition of claim 45, wherein the microbial pathogen is a parasite selected from the group consisting of Plasmodium spp., Leishmania, and a helminth.

49. The composition of claim 45, wherein the microbial pathogen is a fungus selected from the group consisting of Candida spp., Aspergillus spp., Cryptococcus spp., Mucoromycete, Blastomyces dermatitidis, Histoplasma capsulatum, and Sporothrix schenckii.

50. The composition of any one of claims 40-43, wherein the antigen is a cancer-specific antigen.

51. The composition of claim 50, wherein the antigen is a heteroclitic epitope or a cryptic epitope derived from the cancer-specific antigen.

52. The composition of claim 50, wherein the cancer-specific antigen is a neoantigen.

53. The composition of claim 40, wherein the antigen is a psychoactive substance or its hapten derivative (e.g., an opioid-specific antigen).

54. The composition of claim 40, wherein the antigen is a hapten.

55. The composition of claim 40, wherein the antigen comprises a lipopolysaccharide (LPS).

56. The composition of any of claims 40-55, further comprising a pharmaceutically acceptable carrier.

57. The composition of any of claims 40-56, wherein the composition is a vaccine composition.

58. The composition of claim 57, wherein the compound is an adjuvant.

59. The composition of claim 57 or 58, wherein the antigen is adsorbed onto alum.

60. The composition of any of claims 57-59, wherein the compound is adsorbed onto alum.

61. The composition of any of claims 58-60, wherein the vaccine composition further comprises a second adjuvant.

62. The composition of claim 61, wherein second adjuvant is an agonist of Pattern Recognition Receptors (PRRs) selected from the group consisting of Toll-like receptors (TLRs), NOD-like receptors (NLRs), RIG-I-like receptor, C-type Lectin receptors (CLRs), and a stimulator of interferon genes (STING).

63. The composition of claim 61 or 62, wherein second adjuvant is bound to or adsorbed to alum.

64. The composition of any of claims 61-63, wherein the second adjuvant is alum.

65. A vaccine comprising the composition of any of claims 40-64.

66. The vaccine of claim 65, wherein the vaccine is a subunit vaccine, an attenuated vaccine, a conjugate vaccine, or an mRNA vaccine.

67. A method of enhancing an immune response to an antigen in a subject in need thereof, the method comprising administering to the subject an effective amount of the composition of any of claims 40-64.

68. The method of claim 67, wherein the antigen and the compound are administered simultaneously.

69. The method of claim 67 or 68, wherein the antigen and the compound are administered separately.

70. The method of any of claims 67-69, wherein the antigen and the compound are administered once to the subject.

71. The method of any of claims 67-69, wherein the antigen and the compound are administered repeatedly to the subject.

72. The method of any of claims 67-71, wherein the compound and/or the second adjuvant enhance antigen-presenting cell activity.

73. The method of any of claims 67-72, wherein the compound and/or second adjuvant activates B cell immunity.

74. The method of any of claims 67-73, wherein the production of antigen-specific antibodies is increased, compared to when the antigen is administered alone.

75. The method of any of claims 67-74, wherein the activation of antigen-specific cytotoxic T cells is increased, compared to when the antigen is administered alone.

76. The method of any of claims 67-75, wherein the compound polarizes the innate immune response toward T helper 1 (Th1) immunity, T helper 2 (Th2) immunity, T helper 17 (Th17) immunity, and/or T follicular helper (Tfh) cell immunity.

77. The method of any of claims 67-76, wherein the compound prolongs the durability of protection in the subject against the antigen, compared to when the antigen is administered alone.

78. The method of any of claims 67-77, wherein the compound increases the rate of an immune response compared to when the antigen is administered alone.

79. The method of any of claims 67-78, wherein the compound achieves antigen dose sparing such that the antigen induces a same level of immune response at a lower dose in the presence of the compound, compared to when the antigen is administered alone.

80. The method of any of claims 67-79, wherein the subject has or is at risk of developing an infectious disease.

81. The method of claim 80, wherein the infectious disease is caused by a bacterium, a mycobacterium, a fungus, a virus, a parasite or a prion.

82. The method of claim 80 or 81, wherein the infectious disease is sepsis.

83. The method of any of claims 67-79, wherein the subject has or is at risk of developing cancer.

84. The method of claim 83, wherein the cancer is metastatic cancer.

85. The method of claim 83 or 84, wherein the cancer is melanoma.

86. The method of any one of claims 67-79, wherein the subject has or is at risk of developing allergy.

87. The method of any one of claims 67-79, wherein the subject has or is at risk of developing an addiction and/or death due to an overdose of a psychoactive substance.

88. The method of any one of claims 67-87, wherein the administration is systemical or local.

89. The method of any one of claims 67-88, wherein the administration is intramuscular, intradermal, oral, intravenous, topical, transdermal, intranasal, intravaginal, or sublingual.

90. The method of any one of claims 67-89, wherein the administration is prophylactic.

91. The method of any one of claims 67-89, wherein the administration is therapeutic.

92. The method of any one of claims 67-89, wherein the subject is a human infant, an adult, or an older adult.

93. The method of claim 92, wherein the subject is a human infant.

94. The method of claim 93, wherein the human infant is less or equal to 28 days of age or less than or equal to 24 hours of age at the time of administration, or the administration occurs at birth.

95. The method of claim 94, wherein a second administration occurs when the subject is less than or equal to 28 days of age or less than or equal to 6 months of age.

96. The method of claim 93, wherein the administration occurs when the human infant is 2 months, 4 months, and 6 months of age.

97. The method of any one of claims 67-96, wherein the subject is born prematurely or has low birth weight.

98. The method of claim 92, wherein the subject is a human adult.

99. The method of claim 92, wherein the subject is an older adult.

100. The method of claim 99, wherein the administration occurs when the subject is more than 65 years of age.

101. The method of any one of claims 67-100, wherein the subject is immunocompromised due to primary immunodeficiency, acquired immunodeficiency and/or distinct immunity.

102. A method of vaccinating a subject in need thereof, the method comprising administering to the subject an effective amount of the composition of any one of claims 60-63, or the vaccine of any one of claims 64-67.
